(19) United States
(12) Patent Application Publication
(10) Pub. No.: US 2018/0263477 A1
(43) Pub. Date: Sep. 20, 2018

(54) CAMERA SYSTEM FOR MONITORING INSIDE OF BODY

(71) Applicant: Sharp Kabushiki Kaisha, Sakai City, Osaka (JP)

(72) Inventors: Hitoshi AOKI, Sakai City (JP); Toshihisa GOTOH, Sakai City (JP); Kei URAKAWA, Sakai City (JP); Kishoh TAKAMATSU, Sakai City (JP)

(21) Appl. No.: 15/546,291

(22) PCT Filed: Dec. 2, 2015
(86) PCT No.: PCT/JP2015/083930

§ 371 Date: Jul. 26, 2017

(30) Foreign Application Priority Data
Feb. 10, 2015 (JP) 2015-024669

(51) Int. Cl. A61B 1/045; A61B 1/05; A61B 17/34

(52) U.S. Cl. CPC A61B 1/045; A61B 1/05; A61B 90/37; A61B 2017/3445; A61B 17/3421

(57) ABSTRACT

A camera system for monitoring an inside of a body, which reduces an influence of a high-frequency noise, and has high reliability and ease of use is realized. The camera system for monitoring an inside of a body includes a camera support tube (13) formed of a conductive material, a metal spring (191) causing the camera support tube (13) to be grounded, a support-tube joining unit (14) joining the camera unit (11) and the camera support tube (13), and a control system (3) including a display (18).

### TECHNICAL FIELD

The present invention relates to a camera system for monitoring the inside of a body, which includes an imaging unit which can be guided toward the inside of a body.

### BACKGROUND ART

Endoscopic surgery is minimally invasive surgery in which examination or medical treatment is performed without laparotomy with respect to a patient. In the endoscopic surgery, a surgical tool such as forceps, and an endoscope are separately guided toward the inside of a body cavity of the patient. A surgeon captures an image of a tip of the surgical tool inserted into the body cavity within a field of view of the endoscope and performs the treatment operation by using the surgical tool, while observing treatment state of the affected part by using the endoscope. In the endoscopic surgery, the surgical tool and the endoscope are guided toward the inside of the body cavity through a pipe (referred to as a trocar) that is inserted through the abdomen of the patient.

The surgeon, when performing incision or suturing of an organ, enlarges the image by causing the endoscope to approach the organ. Thus, the field of view of the surgeon becomes very narrow. Therefore, an apparatus which can capture a wide region in which a state outside the work region (for example, the movement of the surgical tool, a bleeding site, and a residual item such as gauze, outside a work region) is also observed is required.

In view of such a demand, PTL 1 describes an apparatus that enables a needle-like connector electrode to be directly inserted into an abdominal wall and enables the connector electrode and a camera to be joined to each other in a body.

PTL 2 describes an apparatus that enables a camera unit and a communication cable, which is joined to the camera unit, to be inserted into a trocar, enables a needle to be inserted into an abdominal wall hole, enables the needle and the communication cable to be drawn out from the abdominal wall hole toward the outside of a body in a state where an end portion of the communication cable is hooked onto the needle, and enables the communication cable to be fixed.

### CITATION LIST

### Patent Literature

PTL 1: Japanese Patent No. 4472727 (issued on Jun. 2, 2010)

PTL 2: Japanese Patent No. 4599474 (issued on Dec. 15, 2010)

PTL 3: Japanese Unexamined Patent Application Publication No. 7-111979 (published on May 2, 1995)

### SUMMARY OF INVENTION

### Technical Problem

In PTL 1, since the needle-like connector electrode is directly inserted into the abdominal wall and the connector electrode is joined to the camera in a body, foreign substances may be incorporated into a joining unit between the connector electrode and the camera and an electrical connection may be poor.

In PTL 2, the communication cable is drawn outside a body and is fixed. However, it is not easy to obtain joining strength between the communication cable and the camera unit because of characteristics of the communication cable, and it is also difficult to change the direction of the camera unit from the outside of a body.

Further, in a case using an electronic endoscope device, measures for a high-frequency noise occurring in the electric knife are required. Specifically, a high-frequency noise is mixed in an image signal and thus an image (video) displayed on a monitor is disturbed or displaying of the image (video) is stopped. Accordingly, measures for such an occurrence are required. As the measures, PTL 3 describes a technology of doubly providing an electric shield on an outer circumference of a signal transmission cable. However, in PTL 3, there is a problem in that the signal transmission cable is thick and a wound of the patient is large.

The present invention is to suggest a camera system for monitoring an inside of a body, which has high reliability and ease of use and to suggest the camera system for monitoring an inside of a body, which has a reduced influence of a high-frequency noise.

### Solution to Problem

In order to solve the above-described problems, according to an aspect of the present invention, there is provided a camera system for monitoring an inside of a body, which includes a support tube which has one end portion guided toward an inside of a body and is formed of a conductive material, a ground unit which has at least a portion formed of a conductive material and causes the support tube to be grounded, an imaging unit which is joined to the support tube inside the body, a joining unit that joins the imaging unit and the support tube, a first cable which is connected to the imaging unit and is drawn toward an outside of the body through the support tube, and a control system which is provided on the outside of the body, electrically connected to the first cable, and includes at least a display device.

In order to solve the above-described problems, according to another aspect of the present invention, there is provided a camera system for monitoring an inside of a body, which includes a support tube which has one end portion guided toward an inside of a body, an imaging unit which is joined to the support tube inside the body, a joining unit that joins the imaging unit and the support tube, a first cable which is connected to the imaging unit and is drawn toward an outside of the body through the support tube, and a control system which is provided on the outside of the body, electrically connected to the first cable, and includes at least a display device. The support tube has a gripping unit at a body-outside end portion, and the gripping unit has an outer diameter greater than an inner diameter of the cannula having an annular structure in which the support tube is capable of being inserted into the cannula.

In order to solve the above-described problems, according to still another aspect of the present invention, there is provided a camera system for monitoring an inside of a body, which includes a support tube which has one end portion guided toward an inside of a body, an imaging unit which is joined to the support tube inside the body, a joining unit that joins the imaging unit and the support tube, a first cable which is connected to the imaging unit and is drawn toward an outside of the body through the support tube, and a control system which is provided on the outside of the body, electrically connected to the first cable, and includes at least a display device. A slit is formed in the support tube so as to enable the first cable to pass from a side surface of the support tube to an inside of the support tube. An edge of the slit includes a portion forming a projection, and a portion of the slit, which corresponds to the portion forming the projection, has a width narrower than a diameter of the first cable.

### Advantageous Effects of Invention

According to an aspect of the present invention, there is an effect in that a camera system for monitoring an inside of a body, which has high reliability and ease of use, is suggested and an influence of a high-frequency noise is reduced in the camera system for monitoring an inside of a body.

### BRIEF DESCRIPTION OF DRAWINGS

FIG. 1(a) is a sectional view schematically illustrating a camera support tube, a camera-side cable, and a support-tube joining unit in a camera system for monitoring an inside of a body according to Embodiment 1, FIG. 1(b) is a sectional view illustrating a joined state of the camera support tube and the support-tube joining unit, FIG. 1(c) is a sectional view taken along line AA in FIG. 1(b), and FIG. 1(d) is a sectional view illustrating a joined state of the camera support tube and a camera unit.

FIG. 2 is a schematic diagram illustrating a configuration of the camera system for monitoring an inside of a body, according to Embodiment 1.

FIG. 3(a) is a sectional view schematically illustrating the camera unit according to Embodiment 1, and FIG. 3(b) is a top view of the camera unit.

FIG. 4(a) is a sectional view illustrating a structure of a cannula, FIG. 4(b) is a sectional view illustrating a state where the camera support tube in FIG. 1 is inserted into the cannula in FIG. 4(a), and FIG. 4(c) is a sectional view illustrating a joined state of the camera support tube inserted into the cannula and the camera unit in FIG. 3.

FIGS. 5(a) to 5(g) are schematic diagrams illustrating a method of installing the camera unit inside a body in Embodiment 1.

FIG. 6 is a schematic diagram illustrating a method of using the camera unit in Embodiment 1.

FIG. 7 is a schematic diagram illustrating a modification example of the camera unit in Embodiment 1.

FIG. 8 is a schematic diagram illustrating a configuration of a camera system for monitoring an inside of a body, according to Embodiment 2.

FIG. 9(a) is a perspective view illustrating a camera support tube according to Embodiment 2, FIG. 9(b) is a perspective view illustrating a joined state of a camera-side cable connector and a device-side cable connector according to Embodiment 2, and FIG. 9(c) is a perspective view illustrating a modification example of the camera support tube in Embodiment 2.

FIG. 10 is a schematic diagram illustrating a configuration of a camera system for monitoring an inside of a body, according to a modification example of Embodiment 2.

FIG. 11 is a schematic diagram illustrating a configuration of a camera system for monitoring an inside of a body, according to Embodiment 3.

FIG. 12(a) is a perspective view illustrating a camera support tube according to Embodiment 3, FIG. 12(b) is a perspective view illustrating a cable holder according to Embodiment 3, and FIG. 12(c) is a perspective view illustrating a joined state of the camera support tube and the cable holder according to Embodiment 3.

FIG. 13 is a diagram illustrating a first modification example of Embodiment 3.

FIG. 14 is a diagram illustrating a second modification example of Embodiment 3.

FIG. 15 is a diagram illustrating a third modification example of Embodiment 3.

FIG. 16 is a diagram illustrating a fourth modification example of Embodiment 3.

FIG. 17 is a diagram illustrating a fifth modification example of Embodiment 3.

FIG. 18 is a diagram illustrating a sixth modification example of Embodiment 3.

FIG. 19 is a schematic diagram illustrating a configuration of a camera system for monitoring an inside of a body, according to Embodiment 4.

FIGS. 20(a) and 20(b) are sectional views illustrating a joined state of a camera unit and a camera support tube inserted into a cannula, and are sectional views illustrating a state where an air plug is provided between an end portion of the camera support tube on an outside of a body and an end portion of the cannula on the outside of the body.

FIG. 21 is a schematic diagram illustrating a configuration of a camera system for monitoring an inside of a body, according to Embodiment 5.

FIG. 22(a) is a perspective view illustrating a camera support tube according to Embodiment 5, FIG. 22(b) is a perspective view illustrating a cable holder according to Embodiment 5, and FIG. 22(c) is a perspective view illustrating a joined state of the camera support tube and the cable holder according to Embodiment 5.

FIG. 23 is a schematic diagram illustrating a configuration of a camera system for monitoring an inside of a body, according to Embodiment 6.

FIG. 24 is a diagram illustrating a modification example of the camera support tube and the support-tube joining unit according to Embodiments 1 to 6.

FIG. 25 is a diagram illustrating another modification example of the camera support tube according to Embodiments 1 to 6.

FIG. 26(a) is a sectional view illustrating a camera support tube illustrated in FIG. 24, FIGS. 26(b) and 26(c) are sectional views illustrating a camera support tube illustrated in FIG. 25, and FIG. 26(d) is a sectional view illustrating still another modification example of the camera support tube.

### DESCRIPTION OF EMBODIMENTS

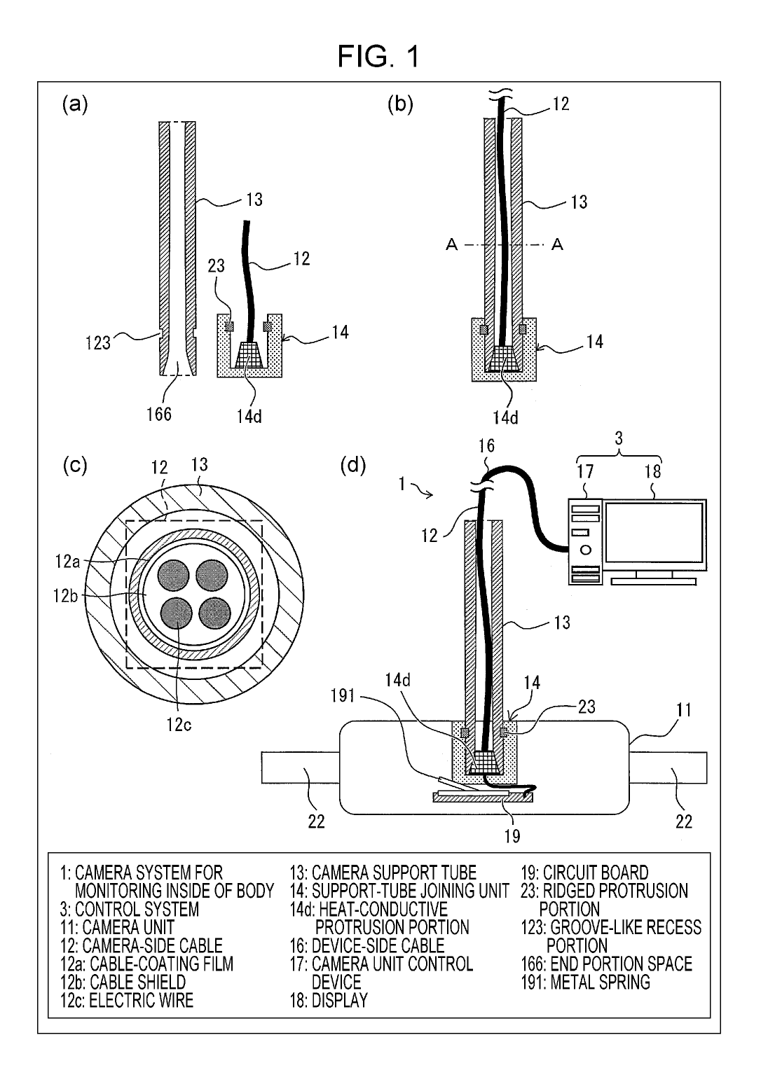

An embodiment of the present invention will be described below with reference to FIGS. 1 to 7. For convenient descriptions, members having the same function as those described in embodiments are denoted by the same reference signs and descriptions thereof will be adequately omitted. A shape of a component illustrated in the drawings and dimensions of the component, such as a length, a size, and a width are not equivalent to an actual shape or actual dimensions. The shape and the dimensions are appropriately changed in order to clarify and simplify the drawings.

### Embodiment 1

(Configuration of Camera System for Monitoring Inside of Body)

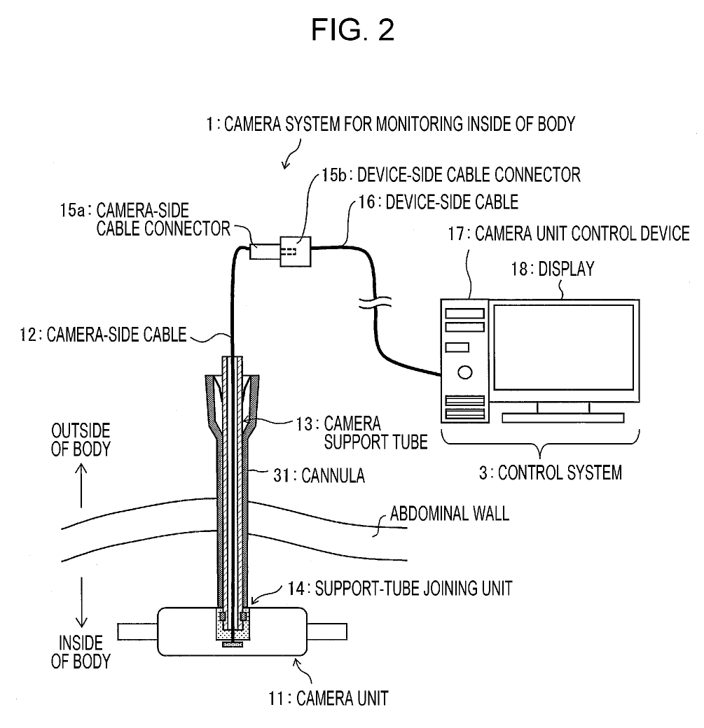

FIG. 2 is a schematic diagram illustrating a configuration of a camera system for monitoring the inside of a body, according to Embodiment 1. As illustrated in FIG. 2, the camera system 1 for monitoring the inside of a body includes an image capturing device, a camera support tube (support tube) 13, a device-side cable connector (second connector) 15b, a device-side cable (second cable) 16, and a control system. The image capturing device includes a camera unit (imaging unit) 11, and a camera-side cable (first cable) 12 and a camera-side cable connector (first connector) 15a which are connected to the camera unit 11. The control system includes a camera unit control device 17 and a display (display device) 18. The cable connectors may be collectively referred to as “a cable connector 15” below.

One end portion of the camera support tube 13 is guided toward the inside of a body through the inside of a cannula 31 which has been made to puncture through an abdominal wall. The camera unit 11 that captures an image of the inside of a body is guided toward the inside of the body through a tube-like member referred to as a trocar. One end portion (on the inside of the body) of the camera support tube 13 and the camera unit 11 which is inside the body are joined to each other at a support-tube joining unit (joining unit) 14 in a state where the camera-side cable 12 passes through the inside of the camera support tube 13. Details of the camera support tube 13 will be described later.

The camera unit 11 is connected to the camera unit control device 17 via the camera-side cable 12, the camera-side cable connector 15a, the device-side cable connector 15b, and the device-side cable 16. An image captured by the camera unit 11 is transmitted to the camera unit control device 17 and a control signal from the camera unit control device 17 is transmitted to the camera unit 11. Specifically, the camera-side cable 12 is connected to the device-side cable 16 in a manner that the camera-side cable connector 15a provided at an end portion of the camera-side cable 12 on a side opposite to that on which the camera-side cable 12 is connected to the camera unit 11 is fitted to the device-side cable connector 15b provided at an end portion of the device-side cable 16 on a side opposite to that on which the device-side cable 16 is connected to the camera unit control device 17. For example, as illustrated in FIG. 2, both of the cable connectors are fitted together by inserting a pin portion of the male type camera-side cable connector 15a into the female type device-side cable connector 15b. Thus, the camera unit 11 and the camera unit control device 17 are connected to each other. The male type and the female type may be reversed, and a configuration in which the female type camera-side cable connector 15a is fitted to the male type device-side cable connector 15b may be made.

Although detailed descriptions will be made later, when the camera unit 11 and the camera support tube 13 are connected to each other, the camera-side cable connector 15a and the camera-side cable 12 are drawn from the inside of the body toward the outside of the body through the camera support tube 13. Thus, the outer diameter of the camera-side cable connector 15a is smaller than the outer diameter of the camera support tube 13. Therefore, if the outer diameter of the camera-side cable connector 15a is set to be small, it is possible to reduce the outer diameter of the camera support tube 13. Accordingly, there is a special effect in that minimal invasiveness is improved. That is, it is desirable that the outer diameter of the camera-side cable connector 15a be set to be as small as possible. For example, as illustrated in FIG. 2, it is desirable that the outer diameter of the camera-side cable connector 15a be set to be equal to or smaller than the outer diameter of the device-side cable connector 15b.

In FIG. 2, the outer diameter of the camera-side cable connector 15a is illustrated as being larger than the actual outer diameter in order to make the drawings easy to understand. In practice, as described above, the outer diameter of the camera-side cable connector 15a is smaller than the outer diameter of the camera support tube 13. FIG. 2 illustrates one pin portion for simplification. Generally, the number of pins corresponds to the number of electric wires used in the cable. This is also similar in other drawings in which the camera support tube 13 and the camera-side cable connector 15a are illustrated.

Since a wired method is employed in transmission from the camera unit 11 to the camera unit control device 17, it is possible to increase the transmission speed, and to stably transmit and receive a signal. Thus, it is possible to obtain an image having high resolution. In addition, it is possible to perform communication at a power lower than that in a wireless method, and to reduce the size of the camera unit 11 by supplying a power from the outside. Accordingly, since the size thereof is reduced, and thus it is possible to minimize a wound occurring when the camera unit 11 is guided toward the inside of the body, there is a special effect that minimal invasiveness is improved.

The camera unit control device 17 displays an image transmitted from the camera unit 11, on the display 18 and transmits a control signal to the camera unit 11. The camera unit control device 17 and the display 18 may be integrally or separately provided.

(Configuration of Image Capturing Device)

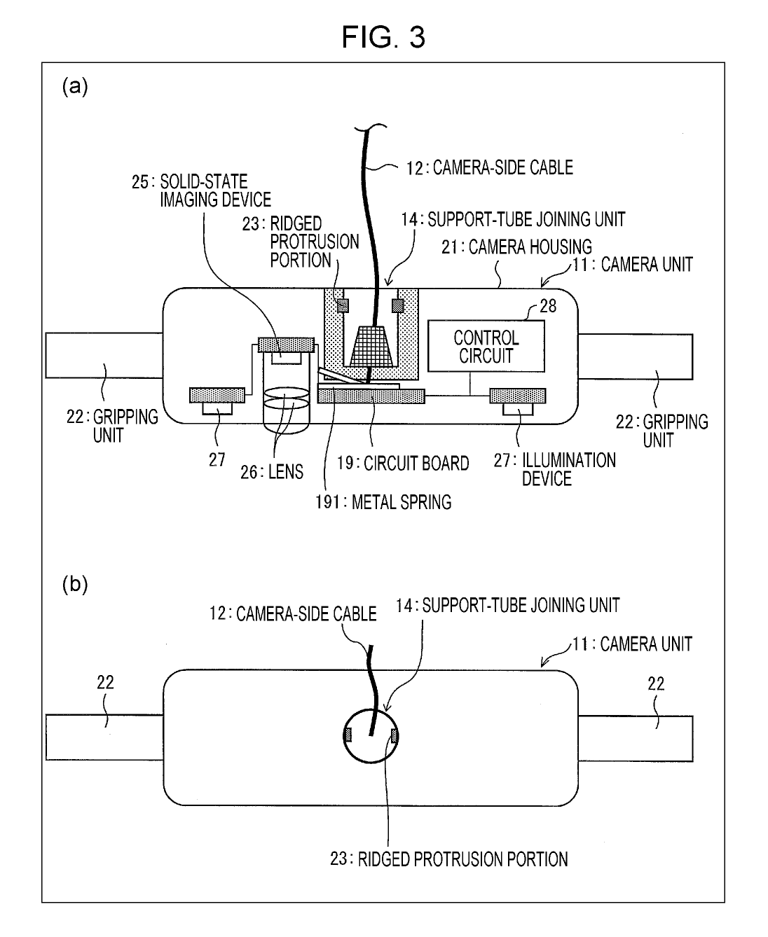

FIG. 3(a) is a sectional view schematically illustrating the camera unit in Embodiment 1. FIG. 3(b) is a top view of the camera unit. As illustrated in FIGS. 3(a) and 3(b), in the camera unit 11, a circuit board 19, a solid-state imaging device 25, a control circuit 28, an illumination device 27, and a lens 26 are provided in a camera housing 21. The solid-state imaging device 25, the control circuit 28, and the illumination device 27 are connected to the circuit board 19. A recess-like support-tube joining unit 14 is provided on an upper surface of the camera housing 21. The support-tube joining unit 14 has a cyclic opening hole structure. A ridged protrusion portion 23 which goes around the inner side surface of the support-tube joining unit 14 is provided on the inner side surface thereof. Gripping units 22 protrude from each of both of the opposing side surfaces of the camera housing 21. The gripping unit 22 is gripped by using forceps when the camera unit 11 is guided to the inside of the body. In addition, the gripping unit 22 is gripped by using forceps, so as to cause the upper surface of the camera unit 11 (surface of the camera unit 11 on which the support-tube joining unit 14 is provided) to face the end portion of the camera support tube 13 when the camera unit 11 is joined to the camera support tube 13.

The camera-side cable 12 is connected to the circuit board 19, and is drawn out of the camera unit 11 through the inside of the support-tube joining unit 14. A connection portion of the circuit board 19 and the camera-side cable 12 is sealed by resin and the like. Further, at a part of the inner portion of the support-tube joining unit 14, at which the camera-side cable 12 is drawn out (the bottom portion of the recess-like support-tube joining unit 14), the camera-side cable 12 is adhered and fixed to the inner portion of the support-tube joining unit 14 (for example, the camera-side cable 12 is sealed and fixed to the inner portion thereof by an adhesive or an O ring). Thus, infiltration or incorporation of foreign substances from this portion (into the camera unit 11) is prevented. The camera-side cable 12 is formed of a flexible material in order to be capable of being guided into a body cavity through the trocar.

The solid-state imaging device 25 is a CCD, a CMOS imaging sensor, or the like. The illumination device 27 makes an image captured by the camera unit 11 brighter by lighting the inside of the body. The size of the illumination device 27 is preferably small. For example, a LED or the like can be suitably used as the illumination device 27. As illustrated in FIG. 2, a plurality of illumination devices 27 may be installed in the camera unit 11.

The recess-like support-tube joining unit 14 has a heat-conductive protrusion portion 14d at the bottom portion. The heat-conductive protrusion portion 14d is formed, for example, of a metal material such as a stainless used steel (SUS), which has excellent heat conductivity. The camera-side cable 12 is adhered and fixed to the inside of the heat-conductive protrusion portion 14d. In this case, the camera-side cable 12 is drawn out from the heat-conductive protrusion portion 14d of the support-tube joining unit 14. An example of adhering and fixing includes sealing and fixing using pressure, an adhesive or an O-ring. Infiltration and incorporation of foreign substances into the camera unit 11 from the adhered and fixed portion are prevented. Details of the heat-conductive protrusion portion 14d will be described later.

Further, the camera unit 11 includes a metal spring (ground unit) 191 between the circuit board 19 and the support-tube joining unit 14. The material of the metal spring 191 is not particularly limited as long as the material is a metal having elasticity and having as much of a function of a spring as can be realized. Details of the metal spring 191 will be described later.

A portion of the camera housing 21 in the camera unit 11, at which the lens 26 or the illumination device 27 is disposed is formed of a transparent material. However, it is desirable that other portions of the camera housing 21 be formed of a material having a blue color or a green color which is easily noticed on the inside of the body. It is more desirable that a film on the surface of the camera-side cable 12 be formed of a material having a blue color or a green color. Further, it is desirable that the camera-side cable connector 15a be also formed of a material which is similarly colored. As described above, since a blue color or a green color which is a complementary color with respect to the color of the inside of the body which is red or yellow, visual recognition can be easily performed when installation work or collection work which will be described later are performed on the inside of the body. Regarding portions of the camera-side cable 12, the camera-side cable connector 15a, and the camera housing 21, other than the portion at which the lens 26 or the illumination device 27 is installed, only a part, not the entirety thereof may be colored.

As described above, in addition to a case when coloring is performed with a blue color or a green color, a light-storing material or a reflective material which are likely to be visually recognized may be used as a material used in the surface of the portions of the camera-side cable 12, the camera-side cable connector 15a, and the camera housing 21, other than the portion at which the lens 26 or the illumination device 27 is installed. According to this, when an object is in a shadow of an organ which is not easily visually recognized, or is at an end of a visual field which illumination light does not easily reach, it is possible to directly find the object. Thus, the light-storing material or the reflective material is particularly effective.

(Structure of Camera Support Tube and Cable)

FIG. 1 illustrates a schematic structure of the camera-side cable 12, the camera support tube 13, and the support-tube joining unit 14. FIG. 1(a) is a sectional view schematically illustrating the camera support tube 13, the camera-side cable 12, and the support-tube joining unit 14. FIG. 1(b) is a sectional view illustrating a joined state of the camera support tube 13 and the support-tube joining unit 14. FIG. 1(c) is a sectional view taken along line AA in FIG. 1(b). FIG. 1(d) is a sectional view illustrating a joined state of the camera support tube 13 and the camera unit 11.

The camera support tube 13 is a cylindrical tube. As illustrated in FIG. 1(a), the camera support tube 13 has a groove-like recess portion 123 at an end portion on a side on which the camera support tube 13 is guided toward the inside of the body. The groove-like recess portion 123 goes around an outer side surface. The support-tube joining unit 14 includes the ridged protrusion portion 23 and the heat-conductive protrusion portion 14d which are described above. The camera support tube 13 has a cylindrical shape. Thus, the camera support tube 13 is strong against a physical impact and is easy to assemble using a general cannula which is similarly a cylindrical tube.

As illustrated in FIG. 1(a), the heat-conductive protrusion portion 14d has a shape of a truncated cone which is tapered when approaching the opening portion (inlet unit) of the support-tube joining unit 14. The camera-side cable 12 passes through the hole formed in the axial direction, and thus the camera-side cable 12 and the heat-conductive protrusion portion 14d are adhered and fixed to each other in the hole. Regarding the end part (on the inside of the body) of the camera support tube 13 joined to the support-tube joining unit 14, the inside (end portion space 166) thereof has a reverse tapered shape (a shape of which the inner diameter increases while approaching the tip) which corresponds to the shape of a truncated cone of the heat-conductive protrusion portion 14d. According to this, when the camera support tube 13 is joined by using the camera-side cable 12 as a guide, the reverse tapered end portion space 166 of the camera support tube 13 is induced to the heat-conductive protrusion portion 14d of the support-tube joining unit 14. Thus, it is easy to insert the camera support tube 13.

As illustrated in FIG. 1(b), when the camera support tube 13 is fitted to the support-tube joining unit 14, the outer circumferential surface of an end portion of the camera support tube 13 comes into contact with the inner side wall of the support-tube joining unit 14, and the inner circumferential surface of the end portion of the camera support tube 13 comes into contact with the heat-conductive protrusion portion 14d of the support-tube joining unit 14. Thus, a special effect in that the joining properties of both of the camera support tube 13 and the support-tube joining unit 14 increases, and the heat dissipation properties of heat transferred to the camera support tube 13 from the camera unit 11 is more improved is obtained.

In a case where the end portion space 166 of the camera support tube 13 has a reverse tapered shape, it is desirable that the outer diameter of the camera support tube 13 be set not to be increased in a manner that the outer diameter of the camera support tube 13 is set to be constant, or be set to have a slightly tapered shape and cause the thickness of the camera support tube 13 to be decreased toward the tip. According to this, when the camera support tube 13 is inserted into the tube-like device, such as a cannula, it is possible to avoid an occurrence of a situation where the camera support tube 13 is hooked to the inner wall of the tube-like device and thus is not extracted.

The camera support tube 13 according to the embodiment is formed of a conductive material. Specifically, the camera support tube 13 is formed of a stainless used steel (SUS). Thus, the camera support tube 13 is electrically shielded. If the camera support tube 13 is grounded, it is possible to prevent mixing of high-frequency noise from the electric knife into a signal passing in the camera-side cable 12. The material of the camera support tube is not limited to the SUS as long as the material is a conductive material. For example, the camera support tube may be formed of a metal material other than the SUS.

As illustrated in FIG. 1(c), in the camera-side cable 12, an electric wire 12c is covered with a cable shield 12b and a cable-coating film 12a which function as an electrical shield. The camera support tube 13 formed of a conductive material is positioned on an outside of the camera-side cable 12. Thus, in the camera system 1 for monitoring an inside of a body, according to the embodiment, it is possible to realize a doubled electrical shield.

As illustrated in FIG. 1(d), if the camera support tube 13 and the camera unit 11 are joined to each other, the camera support tube 13 and the circuit board 19 are electrically connected via the above-described metal spring 191. Specifically, in FIG. 1(d), the heat-conductive protrusion portion 14d which is in contact with the camera support tube 13 penetrates the bottom portion of the support-tube joining unit 14, and is brought into contact with the metal spring 191. Here, since the circuit board 19 is grounded, the high-frequency noise flows to the electrical ground from the camera support tube 13 via the metal spring 191, the heat-conductive protrusion portion 14d, and the circuit board 19. Accordingly, it is possible to prevent mixing of a high-frequency noise from the electric knife into a signal passing in the camera-side cable 12, and it is possible to prevent a situation in which an image is disturbed or displaying of an image is stopped by inputting the high-frequency noise to the camera unit control device 17.

The configuration in which the camera support tube 13 and the circuit board 19 are electrically connected to each other is not limited to the above-described example. For example, the support-tube joining unit 14 may be formed of a conductive material such as a metal, and the support-tube joining unit 14 and the circuit board 19 may be brought into contact with the metal spring 191, and thereby the camera support tube 13 and the circuit board 19 may be electrically connected to each other. In this case, the heat-conductive protrusion portion 14d may be omitted. However, in order to improve conductivity and heat-transferring properties, it is preferable that both of the heat-conductive protrusion portion 14d and the support-tube joining unit 14 formed of a conductive material are used.

The component configured to electrically connect the camera support tube 13 and the circuit board 19 is not limited to the metal spring 191. However, a special effect in that it is possible to electrically connect the camera support tube 13 and the circuit board 19 with reliability even if a distance between the support-tube joining unit 14 (or end portion of the heat-conductive protrusion portion 14d on the circuit board 19 side) and the circuit board 19 varies is obtained by using the metal spring 191.

(Insertion of Camera Support Tube into Cannula and Joining to Camera Unit)

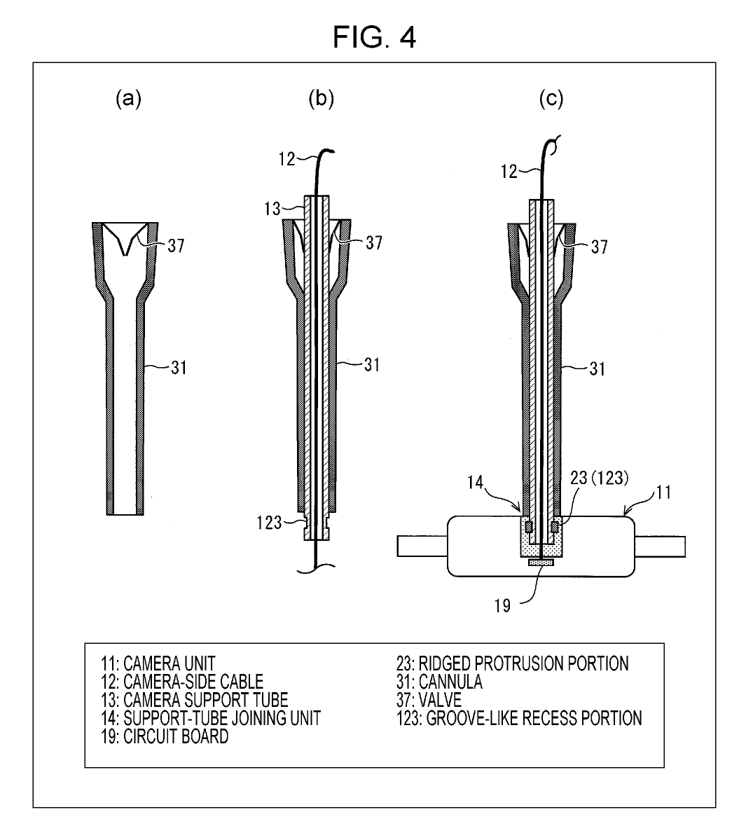

FIG. 4(a) is a sectional view illustrating the cannula. As illustrated in FIG. 4(a), the cannula 31 is a tube-like device. The cannula 31 has a structure in which one end portion (on the outside of the body) is thicker than the other end portion (on the inside of the body), and a valve 37 having restoration properties is provided in the one end portion (on the outside of the body). The valve 37 has a valve structure in which the valve is pressingly expanded when an external force is applied toward the thin end part (on the inside of the body) from the thick end part (on the outside of the body) at the center portion thereof.

In a case where the camera unit 11 is joined to the camera support tube on the inside of the body, firstly, as illustrated in FIG. 4(b), in a state of passing through the camera-side cable 12 inside the camera support tube 13, one thin end portion of the camera support tube 13 is pushed against the thick end portion (on the outside of the body) of the cannula 31, and then the camera support tube 13 is inserted into the cannula 31 until the thin end portion of the camera support tube 13 is exposed from the cannula 31. At this time, the valve 37 is pressingly expanded by the camera support tube 13, and the camera support tube 13 is strongly fastened by the restoration properties. As a result, the camera support tube 13 is fixed to the cannula 31. The thick end portion (on the outside of the body) of the camera support tube 13 is also exposed from the cannula 31. Then, as illustrated in FIG. 4(c), the thin end portion (on the inside of the body) of the camera support tube 13 is inserted into the recess-like support-tube joining unit 14 by using the camera-side cable 12 as the guide. Thus, the ridged protrusion portion 23 is fitted to the groove-like recess portion 123 and the camera unit 11 and the camera support tube 13 are joined to each other at high mechanical strength. In addition, the ridged protrusion portion 23 and the groove-like recess portion 123 may have any shape as long as the portions 23 and 123 are fitted together. Instead of the ridged protrusion portion 23, an O ring and the like may be used.

Strength at which the camera support tube 13 and the support-tube joining unit 14 are fitted together is desirably set to be smaller than adhesion strength of an adhering and fixing portion at which the camera-side cable 12 and the camera unit 11 are adhered and fixed to each other. The reason is as follows. When the camera support tube 13 is inserted into the support-tube joining unit 14 of the camera unit 11, it is necessary that the camera support tube 13 is inserted while the cable is pulled and supported. If fitting strength between the camera support tube 13 and the support-tube joining unit 14 is larger than the adhesion strength of the adhering and fixing portion, the adhering and fixing portion may be damaged.

For example, specifically, strength when the camera support tube 13 and the support-tube joining unit 14 are fitted together is desirably set to be equal to or smaller than 30 N (Newton) which is smaller than the adhesion strength of the adhering and fixing portion. Further, it is desirable that the optimum range be set to be in a range of 3 N to 6 N. If this range is set, it is possible to perform fitting without applying an excessively large force when performing the fitting, and the feeling that the camera support tube 13 is fitted is transferred to the hand. Thus, a special effect that the installation can be performed safely without keeping applying an excessive force is obtained.

### Method and Effect of Using Camera System for Monitoring Inside of Body in Embodiment 1

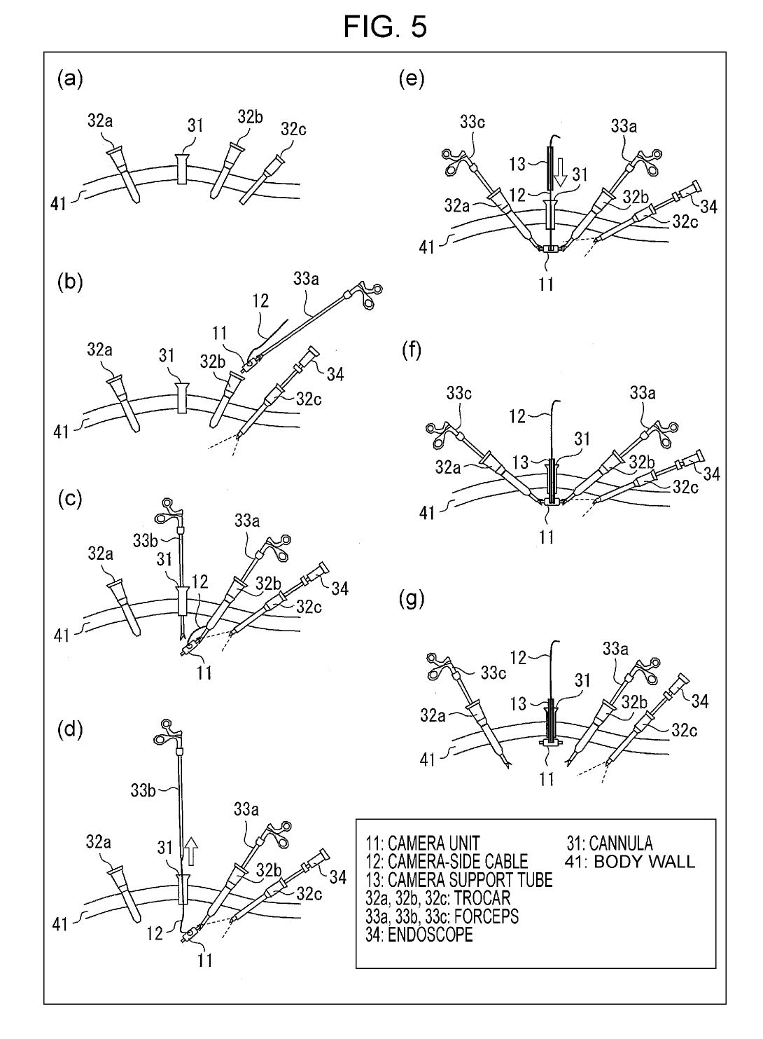

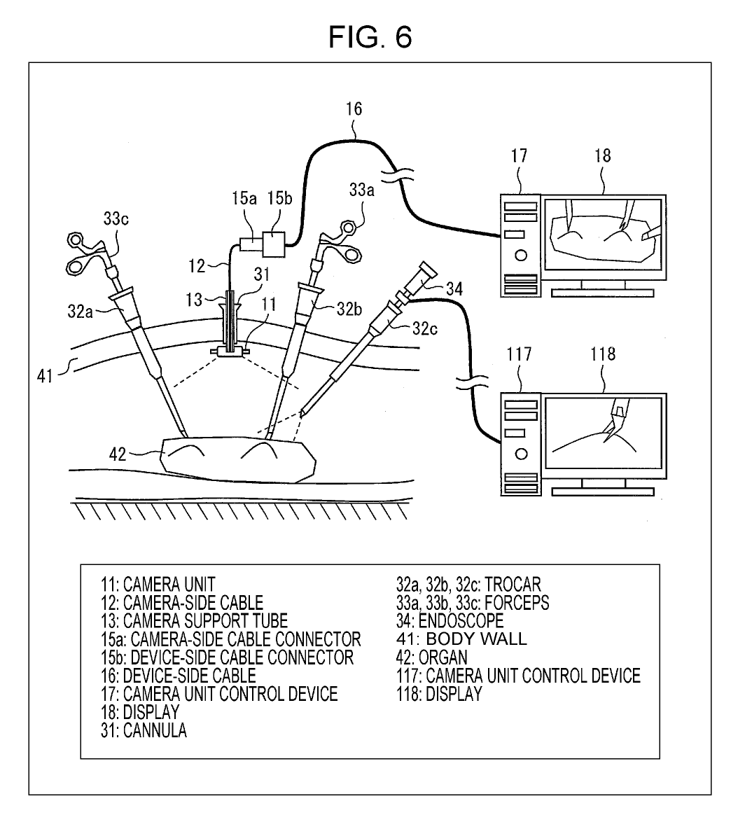

FIGS. 5(a) to 5(g) are schematic diagrams illustrating a method of installing the camera unit inside the body in Embodiment 1. FIG. 6 is a schematic diagram illustrating a use situation of the camera system for monitoring an inside of a body in Embodiment 1.

As illustrated in FIG. 5(a), firstly, a surgeon opens a hole (port) for inserting the forceps or the endoscope into the body cavity on the body wall 41. Then, the surgeon inserts the trocars 32a to 32c to the port. In order to install the camera unit 11 in the body cavity, a port is opened at a position at which the entire organ including an affected portion can be seen, on the body wall 41. Then, the cannula 31 is inserted. Specifically, a needle-like obturator (not illustrated) is made to puncture to the port in a state where the obturator has passed through the inside of the cannula 31, and thus the cannula 31 is inserted into the body wall 41. The diameter of the cannula 31 is preferably small in order to realize minimal invasiveness. Specifically, the diameter of the cannula 31 is preferably is equal to or less than 3 mm. After at least one of the trocar 32a to 32c and the cannula 31 is inserted, the surgeon sends a gas to the inside of the body through the trocar so as to prepare expansion of the inside of the body cavity. Thus, a space for inserting a tool is secured.

Then, as illustrated in FIG. 5(b), the surgeon inserts the endoscope 34 into the body cavity through the trocar 32c. While observing the inside of the body by using the endoscope 34, the surgeon inserts the camera unit 11 which has been gripped by the forceps 33a, into the body cavity through the trocar 32b.

Then, as illustrated in FIG. 5(c), the surgeon operates the forceps 33a to move the camera unit 11 to the vicinity of the cannula 31, and inserts the forceps 33b into the body cavity through the cannula 31.

Then, as illustrated in FIG. 5(d), the surgeon picks off the forceps 33b from the cannula 31 in a state where the camera-side cable 12 is nipped by the forceps 33b. Thus, the camera-side cable 12 is guided toward the outside of the body. At this time, the camera unit 11 (gripping unit 22 thereof) is in a state of being gripped by the forceps 33a.

Then, as illustrated in FIG. 5(e), the surgeon inserts the forceps 33c into the body cavity through the trocar 32a. While the gripping units 22 on both side surfaces of the camera unit 11 are gripped by using the two forceps 33a and 33c so that the support-tube joining unit 14 of the camera unit 11 and the opening of the cannula 31 are parallel and close to each other, the surgeon causes the camera-side cable 12 guided toward the outside of the body to pass through the inside of the camera support tube 13, and inserts the camera support tube 13 into the cannula 31.

Then, as illustrated in FIG. 5(f), the surgeon inserts the end portion of the camera support tube 13 exposed from the cannula 31 into the support-tube joining unit 14 of the camera unit 11 by using the camera-side cable 12 as the guide, so as to join the camera support tube 13 and the camera unit 11 to each other.

When the camera support tube 13 is inserted into the support-tube joining unit 14, a force (for example, 3 N to 6 N) required for fitting the camera support tube 13 with the support-tube joining unit 14 is set to be sufficiently smaller than the adhesion strength (for example, 30 N or larger) of the adhering and fixing portion of the camera-side cable 12 and the camera unit 11. Thus, it is possible to safely insert and fit the camera support tube 13 by pulling the cable while guiding the cable.

Then, as illustrated in FIG. 5(g), the surgeon pulls the camera support tube 13 up so as to capture an image of the inside of the body cavity as wide as possible, and brings the camera unit 11 into contact with the end portion of the cannula 31 on the inside of the body. Since the camera support tube 13 is strongly fastened by the valve 37 (see FIG. 4) of the cannula 31, the camera support tube 13 and the camera unit 11 maintains this state.

After the camera unit 11 is installed in the body, as illustrated in FIG. 6, the camera-side cable 12 and the device-side cable 16 are joined to each other by using the cable connector 15. Thus, a local image of a treated portion is displayed on the display 118 by an endoscope control device 117, and the entire image of the inside of the organ 42, which is captured by the camera unit 11 is displayed on the display 18 by the camera unit control device 17.

The connection between the camera-side cable 12 and the device-side cable 16 is not limited to the above-described example. Specifically, regarding the connection between the camera-side cable 12 and the device-side cable 16, it is desirable that an intermediate cable (not illustrated) configured to connect the camera-side cable 12 and the device-side cable 16 be provided between these cables 12 and 16. In this manner, it is possible to gradually change the cable diameter of the very fine camera-side cable 12 and the cable diameter of the thick device-side cable 16, or the thickness of the cable connector. In addition, it is possible to cause the necessity of use of the fine cable which is relatively slow at the transmission speed to be the lowest limit. Thus, it is possible to increase the transmission speed and to obtain an image having high resolution. The camera-side cable connector 15a and a first intermediate cable connector (not illustrated) are fitted together, and thus the camera-side cable 12 and the intermediate cable are connected to each other. The device-side cable connector 15b and a second intermediate cable connector (not illustrated) are fitted together, and thus the device-side cable 16 and the intermediate cable are connected to each other. In a case where the cable diameter or the thickness of the cable connector is gradually changed in this example, it is desirable that “the outer diameter of the camera-side cable 12<the outer diameter of the intermediate cable<the outer diameter of the device-side cable 16” and “the outer diameter of the camera-side cable connector 15a the outer diameter of the first intermediate cable connector<the outer diameter of the second intermediate cable connector the outer diameter of the device-side cable connector 15b” be set.

There is a special effect in that separation of a clean field and an unclean field during the surgery is effectively performed by using the intermediate cable. In other words, in order to cause the handling to be easy regarding the above-described transmission speed or during the installation, the camera-side cable 12 to be insert into the inside of the body is set to have a length which is the lowest necessary limit. The intermediate cable on which sterilization treatment has already been performed is used from here until entering the unclean field beyond the clean field. In this manner, the camera-side cable connector 15a and the first intermediate cable connector can be fitted together in the middle of the clean field, and a clean state can be maintained. The second intermediate cable connector is fitted to the device-side cable connector 15b which is in the unclean field. Thus, the second intermediate cable connector becomes unclean and is handled as unclean device. Therefore, it is possible to completely separate the unclean device from the clean device side.

In addition, the sterilization treatment is performed at a portion included in the “clean field” in the camera system for monitoring an inside of a body, and thus cleanness is maintained. A portion included in the “unclean field” is not subjected to the sterilization treatment, or becomes the “unclean field” after the sterilization treatment is performed.

Connection strength (fitting strength when the camera-side cable 12 is connected (fitted) with the intermediate cable or the device-side cable 16 by the cable connector 15 is desirably set to be smaller than the adhesion strength of the adhering and fixing portion at which the camera-side cable 12 and the camera unit 11 are adhered and fixed to each other.

The reason is as follows. When a force which is as large as being not expected in a normal use is applied to the cable, the connection (fitting) by the cable connector 15 is firstly released, and thus there is no concern that the adhering and fixing portion is damaged or the body wall of a patient is damaged by pulling the camera unit 11 in a direction toward the outside of the body. In addition, the stability is improved. It is also possible to prevent an accident caused when the surgeon or an assistant is hooked by the cable and falls down, or the camera unit control device 17 is pulled and dropped from the table.

For example, specifically, strength when the cables are connected (fitted) with each other by the cable connector 15 is desirably set to be equal to or smaller than 30 N (Newton) which is smaller than the adhesion strength of the adhering and fixing portion. Further, it is desirable that the optimum range be set to be in a range of 4 N to 10 N. If this range is set, it is possible to perform the connection without applying an excessively large force when performing the connection. In addition, even when performing the removing, it is not necessary to apply an excessively large force.

If the fitting strength of the device-side cable connector 15b in the unclean field and the second intermediate cable connector or the fitting strength of the device-side cable 16 and the camera unit control device 17 by a cable connector (not illustrated) of the device-side cable 16 on the camera unit control device 17 side is set to be larger than the fitting strength of the camera-side cable connector 15a and the first intermediate cable connector (for example, set to be 50 N to 100 N), when an unexpected force is applied to the cable, the connection (fitting of the camera-side cable connector 15a and the first intermediate cable connector) of the camera-side cable 12 in the clean field and the intermediate cable can be necessarily released in advance. In an opposite case, if, for example, the connection (fitting of the device-side cable connector 15b and the second intermediate cable connector) of the intermediate cable in the unclean field and the device-side cable 16 is previously released, as a reaction, there is a risk that a portion of the intermediate cable, which is in the unclean field, and the second intermediate cable connector enter the clean field. Therefore, releasing the connection in the clean field has a special effect in securing safety during surgery. In addition, in a case where the connection is released in the clean field, and thus the portion of the intermediate cable, which is in the clean field (that is, a portion (clean portion) of the intermediate cable, which has a predetermined length from a fit portion of the camera-side cable connector 15a and the first intermediate cable connector) and the first intermediate cable connector are brought into contact with the unclean field, the intermediate cable may be exchanged with a clean intermediate cable (including a first intermediate cable connector), and thus safety is ensured. In a case where the cable connector is configured of an independent single component, and in a case where the cable connector is brought into contact with the unclean field along with the portion of the intermediate cable, which is in the clean field, the intermediate cable and the cable connector may be exchanged with a clean intermediate cable and a clean cable connector.

It is desirable that the camera-side cable 12 be sufficiently smaller than the length (about 1 m) obtained by adding the lengths of the camera-side cable 12 and the clean portion. Specifically, it is desirable that the camera-side cable 12 be equal to or smaller than the length obtained by adding the lengths of the camera-side cable 12 and the clean portion, that is, equal to or smaller than 50 cm in the maximum. Accordingly, it is possible to prevent the camera-side cable 12 from entering the unclean field.

In the above-described example, a case where the camera-side cable 12 and the device-side cable 16 are connected to each other by the intermediate cable is described. However, even in a case where the camera-side cable 12 and the device-side cable 16 are directly connected to each other, it is desirable that the camera-side cable 12 be sufficiently smaller than the length (about 1 m) obtained by adding the lengths of the camera-side cable 12 and the clean portion. In this case, the clean portion corresponds to a portion of the device-side cable 16, which has a predetermined length from the fit portion of the camera-side cable connector 15a and the device-side cable connector 15b.

Accordingly, the surgeon can perform treatment by using the forceps 33a and the forceps 33c while enlarging and observing a work region (local region) on the display 18, and can also grasp a state (movement of the forceps or the like, a bleeding site, and a residual such as gauze, outside the work region) outside the work region on the display 18.

The camera unit 11 and the camera support tube 13 are joined to each other at high mechanical strength, and thus the support force of the camera unit 11 is higher than that in the related art. Since the camera-side cable 12 is guided toward the outside of the body through the inside of the camera support tube 13, after the camera unit 11 and the camera support tube 13 are joined with each other, a situation in which a load is applied to the camera-side cable 12, or the camera-side cable 12 is exposed in the body or is brought into contact with the body wall 41 does not occur. Thus, reliability (water repellency and antifouling property of the connection portion) of the electrical connection between the camera-side cable 12 and the circuit board 19 is improved. From the above descriptions, it is possible to realize the camera system for monitoring an inside of a body, which has high reliability.

The surgeon can operate the camera support tube 13 in accordance with the situation, so as to change the direction (direction of the visual field) of the camera unit 11. Specifically, the direction of the camera unit 11 can be changed by using the elastic force of the body wall 41 and by inclining the camera support tube 13. At this time, if the surgeon separates the hand from the camera support tube 13, the direction of the camera unit 11 is brought back to the original direction by the elastic force of the body wall 41. Thus, it is possible to improve work efficiency of the surgeon. The cannula 31 and the camera support tube 13 inserted into the cannula 31 are cylindrical tubes. Thus, it is possible to easily rotate the camera support tube 13 in a circumferential direction. Thus, the surgeon can change the direction of the camera unit 11 without applying a load on the body wall 41. The camera support tube 13 is held by the cannula 31, so as to be movable in a longitudinal direction (extension direction of the tube). Thus, the surgeon pushes the camera support tube 13 to the inside of the body or pulls the camera support tube 13 to the outside of the body, and thus can change an imaging zoom without applying a load to the body wall 41. From the above descriptions, it is possible to realize the camera system for monitoring an inside of a body, which has ease of use.

In Embodiment 1, the cannula 31 and the camera support tube 13 are fixed to each other by the valve in the cannula 31. However, in a case of using a general cannula which does not have a valve, the cannula and the camera support tube 13 can be fixed by a tape.

(Separation Between Camera Unit 11 and Camera Support Tube 13)

Next, a method of separating the camera unit 11 from the camera support tube 13 will be described. Firstly, the surgeon pulls the camera support tube 13 in the direction toward the outside of the body, in a state of gripping the gripping unit 22 of the camera unit 11 in the body by using the forceps 33a and the forceps 33c. Then, the surgeon picks off the camera support tube 13 from the support-tube joining unit 14 of the camera unit 11. Then, after picking off the camera support tube 13 from the cannula 31 so as to separate the camera support tube 13 and the camera-side cable 12, the surgeon guides the camera unit 11 and the camera-side cable 12 from the trocar 32a or the trocar 32b toward the outside of the body.

Similarly to a case where the camera unit 11 and the camera support tube 13 are separated, it is desirable that the fitting strength of the camera support tube 13 and the support-tube joining unit 14 be smaller than the adhesion strength of the adhering and fixing portion between the camera-side cable 12 and the camera unit 11. If the fitting strength of the camera support tube 13 and the support-tube joining unit 14 is larger than the adhesion strength of the adhering and fixing portion, it is necessary to apply a large force when the camera support tube 13 is removed from the support-tube joining unit 14. Thus, the adhering and fixing portion may be damaged. For example, if the fitting strength is set to be in a range of 3 N to 6 N, it is possible to remove the camera support tube 13 without applying an excessively large force, and the feeling that the camera support tube 13 is removed is transferred to the hand. Thus, a special effect in that the separation can be performed safely without keeping applying an excessive force is obtained.

The camera-side cable connector 15a passes via the inside of the body when being collected, but there is not a problem because of maintaining cleanness.

### Modification Example of Embodiment 1

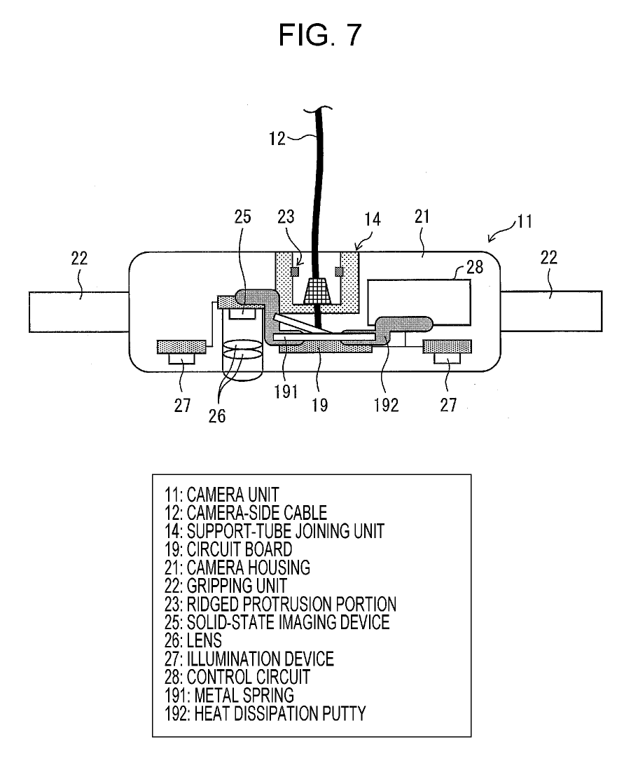

FIG. 7 is a schematic diagram illustrating a modification example of the camera unit 11 in Embodiment 1. As illustrated in FIG. 7, a camera unit 11 according to the modification example links members which emit heat in the camera unit 11 (specifically, circuit board 19, solid-state imaging device 25, and control circuit 28) to the metal spring 191 formed of a conductive material, by using a heat dissipation putty 192. Thus, it is possible to improve the heat dissipation property of the camera unit 11.

In the embodiment, a case where the camera support tube 13 is cylindrical and includes the groove-like recess portion 123 is described. In this embodiment and other embodiments which will be described later, the shape of the camera support tube 13 and the structure of joining the camera support tube 13 with the support-tube joining unit 14 are not limited thereto. For example, the camera support tube 13 may be a tube having a square pillar shape (cross section is rectangular) and a tubular tool such as a trocar or a cannula, which is generally used as the camera support tube may be used as it is. A puncturing device which has a sharp tip or has a shape of being obliquely sliced as an injection needle may be used.

The structure of the camera support tube 13 joining with the support-tube joining unit 14 may be a locking hole provided at a portion (at least two sites) of the outer circumference of the camera support tube 13 or may be a locking male screw. The structure of the support-tube joining unit 14 joining with the camera support tube 13 is changed depending on the structure of the camera support tube 13. That is, in a case where the camera support tube 13 includes a locking hole, the support-tube joining unit 14 includes a locking claw. In a case where the camera support tube 13 includes a locking male screw, the support-tube joining unit 14 includes a locking female screw.

### Embodiment 2

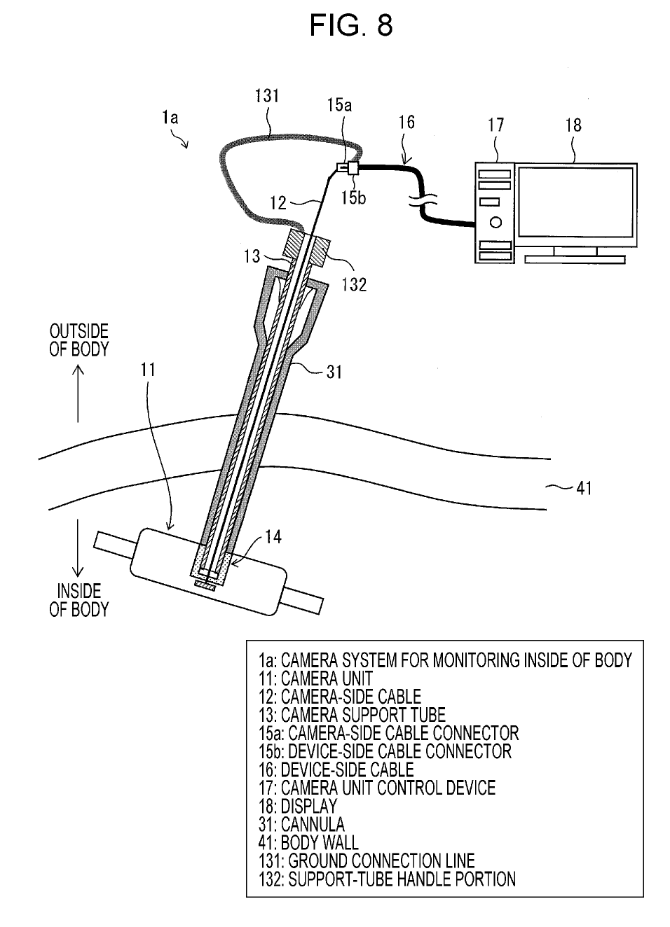

Another embodiment of the present invention will be described based on FIGS. 8 to 10 as follows. For easy description, members having the same function as those described in the above embodiment are denoted by the same reference signs and descriptions thereof will not be repeated.

In Embodiment 1, the camera support tube 13 is grounded in the camera unit 11. In this embodiment, a configuration in which the camera support tube 13 is electrically connected to the grounded cable connector 15, so as to be grounded. FIG. 8 is a schematic diagram illustrating a configuration of a camera system 1a for monitoring an inside of a body, according to this embodiment.

As illustrated in FIG. 8, in the camera system 1a for monitoring an inside of a body, the camera support tube 13 and the device-side cable connector 15b are electrically connected to each other via a ground connection line (ground line) 131. Here, since the device-side cable connector 15b is grounded, a high-frequency noise flows to the ground from the camera support tube 13 via the ground connection line 131 and the device-side cable connector 15b. Accordingly, it is possible to prevent mixing of a high-frequency noise from the electric knife into a signal passing in the camera-side cable 12, and it is possible to prevent a situation in which an image is disturbed or displaying of an image is stopped by inputting the high-frequency noise to the camera unit control device 17.

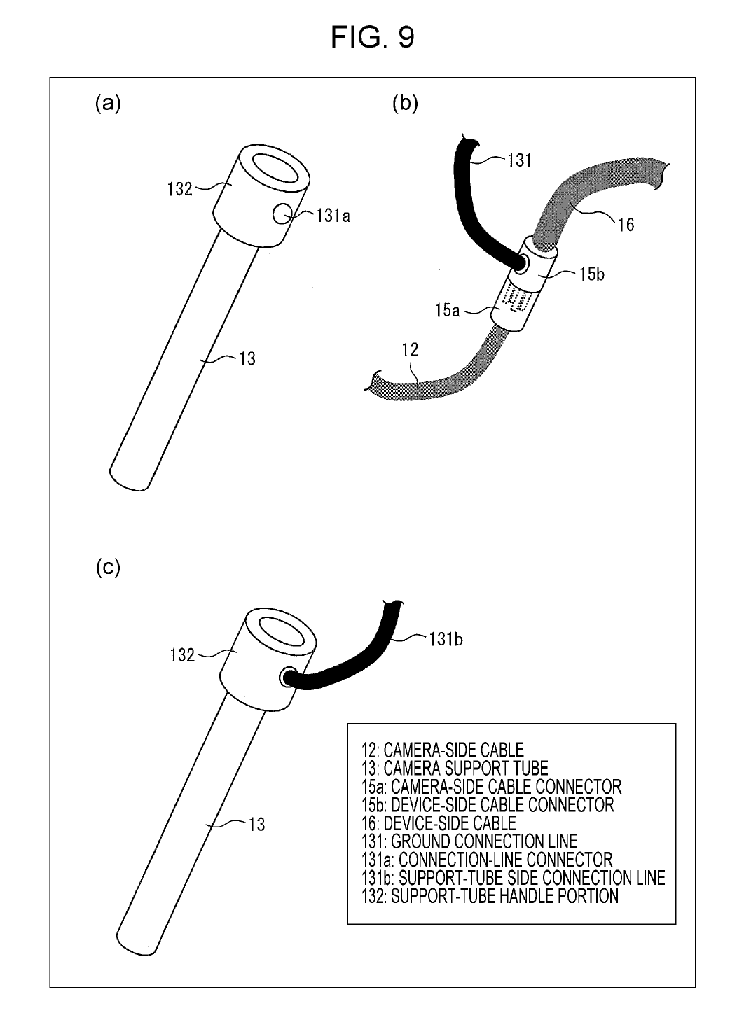

FIG. 9(a) is a perspective view illustrating the camera support tube 13 according to this embodiment. FIG. 9(b) is a perspective view illustrating a joined state of the camera-side cable connector 15a and the device-side cable connector 15b according to this embodiment. FIG. 9(c) is a perspective view illustrating a modification example of the camera support tube 13 in this embodiment.

As illustrated in FIG. 9(a), the camera support tube 13 in this embodiment includes a support-tube handle unit (gripping unit) 132 at an end portion thereof on a side which is not guided toward the inside of the body. The support-tube handle unit 132 has a cylindrical shape in which the diameter of a portion on the side which is not guided toward the inside of the body is longer than that of a portion on a side which is guided toward the inside of the body. The support-tube handle unit 132 functions to prevent falling of the support tube to the inside of the body, and is desirably provided at the end portion of the support tube. In particular, as illustrated in FIG. 8, it is desirable that the support-tube handle unit 132 have an outer diameter greater than the inner diameter of the cannula 31 or the trocar. The camera support tube 13 including such a support-tube handle unit 132 can be also applied to a camera system for monitoring an inside of a body, which does not have an electric shield function.

A connection-line connector (ground-line connector) 131a that connects the ground connection line 131 and the camera support tube 13 is provided in the support-tube handle unit 132. The connection-line connector 131a may have a configuration of being electrically connected with the ground connection line 131. For example, a configuration in which a portion formed of a conductive material is provided in order to fix a lead wire of the ground connection line 131 may be made. The ground connection line 131 is connected to the connection-line connector 131a, and thus it is possible to cause the camera support tube 13 to be grounded and cause the high-frequency noise from the electric knife to flow into the ground.

Regarding a method of connecting the ground connection line 131 and the camera support tube 13, any method is possible as long as the ground connection line 131 and the camera support tube 13 are electrically connected to each other. The method of the connection is not limited to a method of using the above-described connection-line connector 131a. For example, as illustrated in FIG. 9(c), the camera support tube 13 may have a structure of including a support-tube side connection line (support-tube connection line) 131b which is an electric wire used for connection with the ground connection line 131. Here, the ground connection line 131 and the support-tube side connection line 131b may be connected by any method of the connection lines 131 and 131b being electrically connected. For example, conductive connectors (not illustrated) which are respectively provided on the ground connection line 131 and the support-tube side connection line 131b may be connected to each other. In addition, lead wires of the ground connection line 131 and the support-tube side connection line 131b may be exposed and the exposed wires may be connected to each other.

The length of the support-tube side connection line 131b is not particularly limited. It is preferable that the support-tube side connection line has such a length that it does not interfere with the work. Specifically, it is preferable that the support-tube side connection line 131b has such a length that it is not brought into contact with the cannula 31 or the trocar when the camera support tube 13 is inserted into the tube-like member such as the cannula 31 or the trocar.

The camera support tube 13 is not limited to the configurations illustrated in FIGS. 9(a) to 9(c). For example, a camera support tube which does not include the support-tube handle unit 132 and has both end portions having the same diameter may be provided.

As illustrated in FIG. 9(b), the ground connection line 131 is electrically connected to the device-side cable connector 15b. It is preferable that the ground connection line 131 is connected with the device-side cable connector 15b in a state where detachment is not possible, in consideration of the safety. However, it is not limited thereto. The device-side cable connector 15b and the ground connection line 131 may be detachable from each other, and the device-side cable connector 15b and the ground connection line 131 may be connected to each other by a connection-line connector (not illustrated) and the like provided on the device-side cable connector 15b. The connection-line connector has a configuration similar to that of the above-described connection-line connector 131a, for example. However, the configuration of the connection-line connector is not particularly limited as long as the device-side cable connector 15b and the ground connection line 131 can be electrically connected.

### Modification Example of Embodiment 2

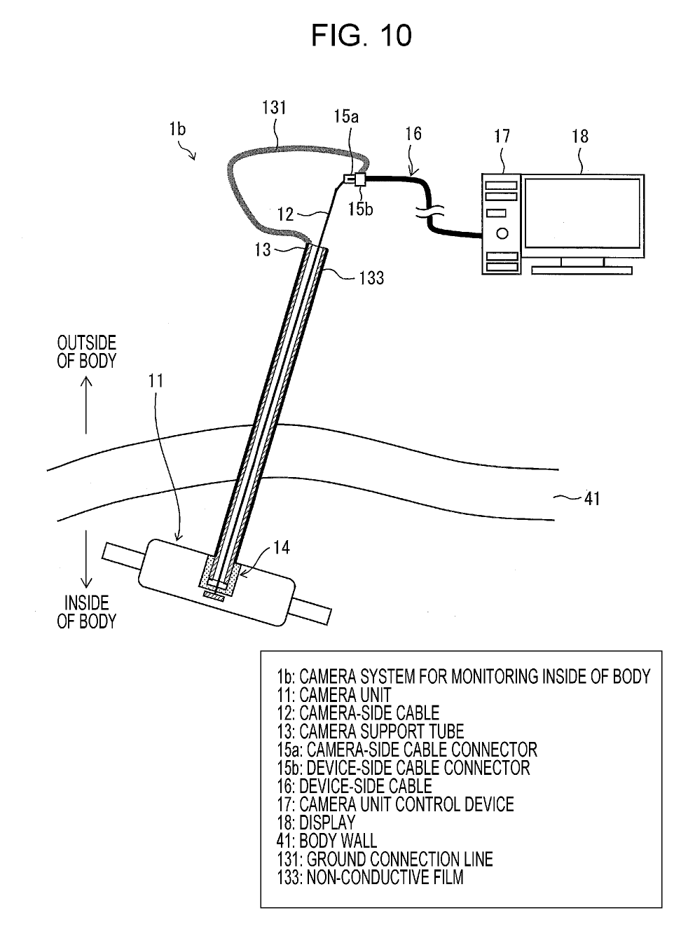

FIG. 10 is a schematic diagram illustrating a configuration of a camera system 1b for monitoring an inside of a body, according to a modification example of the embodiment. The camera system 1b for monitoring the inside of a body is different from the above-described camera system 1a for monitoring the inside of a body, in an aspect that a non-conductive film 133 is provided on the outer circumference of the camera support tube 13. That is, in the modification example, the camera support tube 13 is coated with the non-conductive film 133. Although the non-conductive film 133 is formed, for example, of resin, the material of the film 133 is not limited to the resin if the material is a non-conductive material and is capable of coating the camera support tube 13. Thus, as illustrated in FIG. 10, in a case where the camera support tube 13 is directly guided toward the inside of the body without using the tube-like member such as the cannula 31 or the trocar, the body does not come into contact with the conductive portion (portion in which a current may flow) of the camera support tube 13. Accordingly, it is possible to improve safety for the body.

As illustrated in FIG. 10, it is preferable that a portion of the surface of the camera support tube 13, which is joined to the support-tube joining unit 14, is not coated with the non-conductive film 133. If the above portion is not coated with the non-conductive film 133, it is possible to prevent degradation of the heat-transferring property and conductivity.

### Embodiment 3

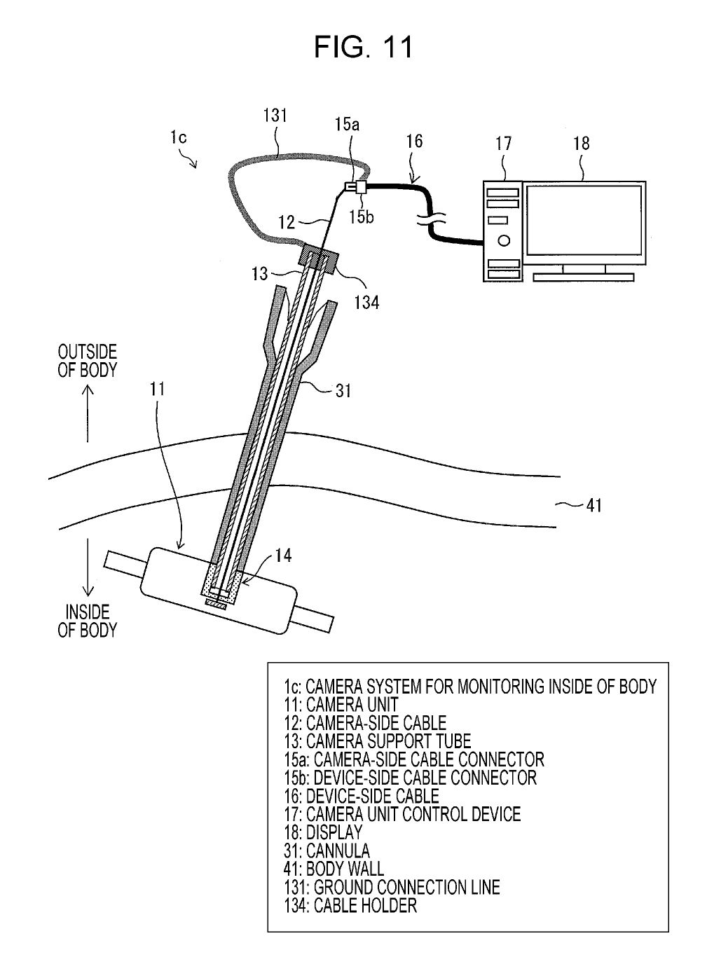

Still another embodiment of the present invention will be described based on FIGS. 11 to 18 as follows. For easy description, members having the same function as those described in the above embodiment are denoted by the same reference signs and descriptions thereof will not be repeated.

FIG. 11 is a schematic diagram illustrating a configuration of a camera system 1c for monitoring an inside of a body, according to this embodiment. In Embodiment 2, the ground connection line 131 is directly electrically connected to the camera support tube 13. In the embodiment, as illustrated in FIG. 11, a configuration in which the ground connection line 131 is connected to a cable holder 134 which is attached to the camera support tube 13 so as to fix the camera-side cable 12 to the camera support tube 13 will be described. In the camera support tube 13 illustrated in FIG. 11, the support-tube handle unit 132 is omitted. However, the camera support tube 13 may include the support-tube handle unit 132.

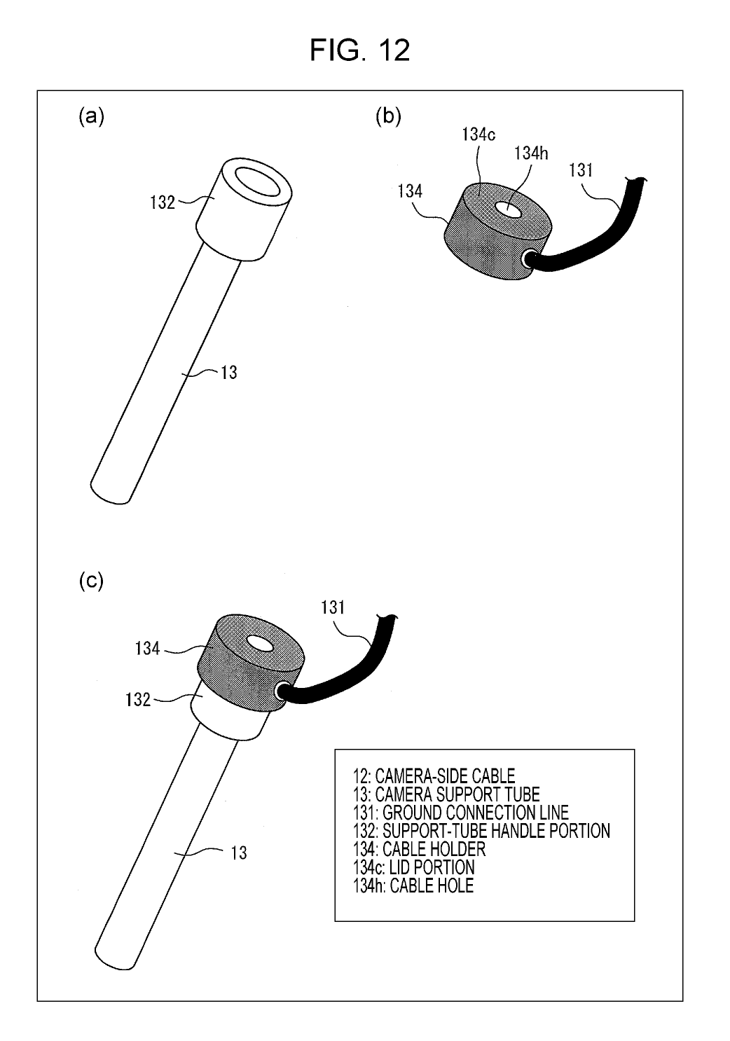

FIG. 12(a) is a perspective view illustrating the camera support tube 13 according to this embodiment. FIG. 12(b) is a perspective view illustrating a cable holder 134 according to this embodiment. FIG. 12(c) is a perspective view illustrating a joined state of the camera support tube 13 and the cable holder 134 according to this embodiment.

As illustrated in FIG. 12(a), the camera support tube 13 according to the embodiment is different from the camera support tube 13 according to Embodiment 2, in an aspect of not including the connection-line connector 131a. As illustrated in FIG. 12(b), the cable holder 134 according to the embodiment has a configuration in which a cable hole 134h which penetrates a lid unit 134c and has a diameter corresponding to the diameter of the camera-side cable 12 is provided. The cable holder 134 is fitted to the camera support tube 13 in a state where the camera-side cable 12 is held in the cable hole 134h, as illustrated in FIG. 12(c). A slit may be provided in the cable holder 134 in order to cause the camera-side cable 12 to easily pass. In addition, the cable holder 134 may have a shape like a clip, which nips and fixes the camera-side cable 12.

The cable holder 134 is configured so as to be connectable with the ground connection line 131 via a connector. The configuration of the connector is not particularly limited as long as the connector can electrically connect the cable holder 134 and the ground connection line 131. For example, the configuration of the connector may be similar to that of the connection-line connector 131a described in Embodiment 2. A portion of the cable holder 134, which comes into contact with the ground connection line 131 and the camera support tube 13, is formed of a conductive material. The portion (referred to as a conductive portion below) formed of the conductive material is configured to electrically connect the camera support tube 13 and the ground connection line 131 (that is, a conductive portion coming into contact with the camera support tube 13 and a conductive portion coming into contact with the ground connection line 131 are integrally formed). Thus, it is possible to cause the high-frequency noise to flow from the camera support tube 13 into the ground via the ground connection line 131 and the device-side cable connector 15b.

As described above, in the embodiment, the cable holder 134 attached to the camera support tube 13 is connected with the ground connection line 131. Thus, it is not necessary that the connection-line connector 131a, the support-tube side connection line 131b, or the like which is described in Embodiment 2 is provided on the camera support tube 13. In other words, it is possible to obtain the measures for the high-frequency noise of the electric knife by using the general camera support tube 13.

In the above-described example, the cable holder 134 is electrically connected to the ground connection line 131 via the connector. However, the cable holder 134 is not limited to the example using the connector, as long as the cable holder 134 and the ground connection line 131 can be electrically connected. For example, an electric wire may be provided in the cable holder 134, and the electric wire and the ground connection line 131 may be connected to each other. It is preferable that the length of the electric wire is as long as does not come into contact with the cannula 31 or the trocar when the camera support tube 13 with which the cable holder 134 is fitted is inserted into the cannula 31 or the trocar.

### Modification Example of Embodiment 3

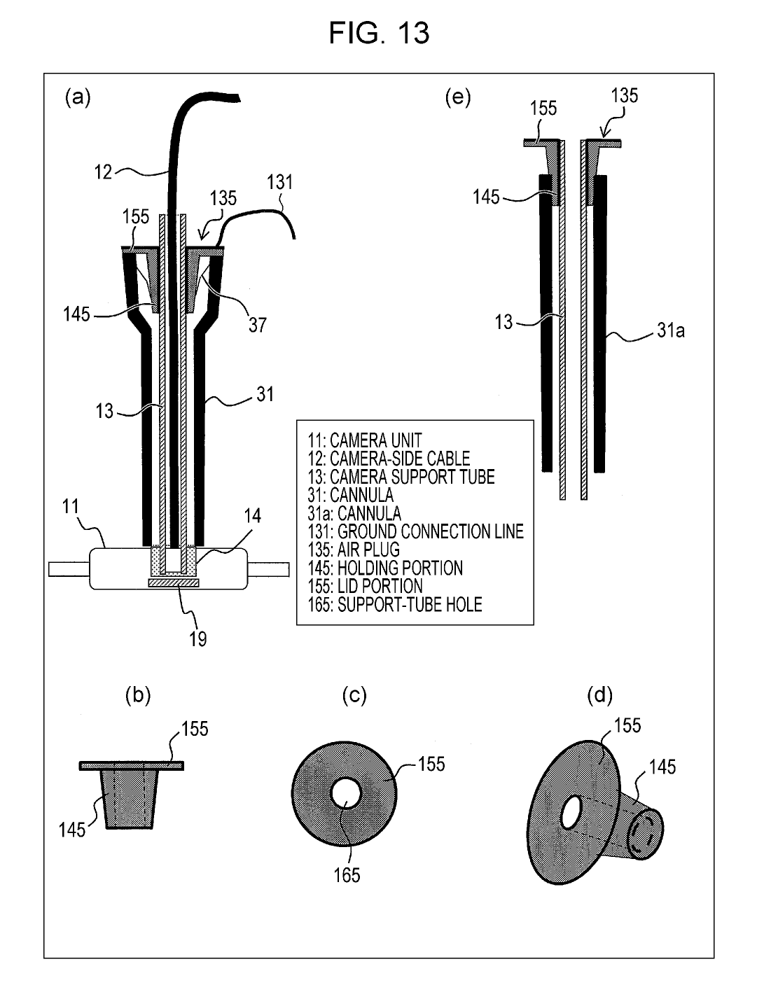

The accessory for the support tube is not limited to the above-described cable holder 134. The accessory for the support tube may be connected to the ground connection line 131 and be attached to the body-outside end portion of the camera support tube 13 or the tube-like member (cannula) such as the cannula 31 or the trocar, so as to come into contact with the camera support tube 13. The tube-like member has an annular structure in which the camera support tube 13 is capable of being inserted into the tube-like member. The function of the accessory for the support tube is not particularly limited. The accessory for the support tube may fix the camera-side cable 12 to the camera support tube 13, like the above-described cable holder 134. The accessory for the support tube may be a so-called a support tube holder that fixes the camera support tube 13 to the tube-like member such as the cannula 31 or the trocar. In addition, the accessory for the support tube may be a so-called air plug that blocks at least a portion of a gap between the camera support tube 13 and the tube-like member. The accessory for the support tube may have plural kinds of functions described above. Some specific examples of such an accessory for the support tube will be described with reference to FIGS. 13 to 18. FIGS. 13 to 18 are diagrams illustrating modification examples of Embodiment 3. In FIGS. 13 to 18, for simple illustrations, the support-tube handle unit 132 is omitted. However, the camera support tube 13 may include the support-tube handle unit 132.

### FIRST MODIFICATION EXAMPLE

Firstly, the accessory for the support tube may be an air plug 135 as illustrated in FIG. 13. FIG. 13(a) is a sectional view illustrating a joined state of the camera support tube 13 inserted into the cannula 31 and the camera unit 11 and is a sectional view illustrating a form in which the air plug 135 is provided between the end portion of the camera support tube 13 on the outside of the body and the end portion of the cannula 31 on the outside of the body. FIG. 13(b) is a side view illustrating the air plug 135. FIG. 13(c) is a top view illustrating the air plug 135. FIG. 13(d) is a perspective view illustrating the air plug 135. FIG. 13(e) is a sectional view illustrating a case of using a cannula 31a in which the size of the end portion on the outside of the body is small.

The tube-like member such as the cannula or the trocar, in which the camera support tube 13 is installed, has various types of internal structures in an aspect of an inner diameter size, a valve, or the like. Thus, when the camera support tube 13 is installed in the tube-like member, inconvenience, for example, a situation in which a gas of expanding the abdominal wall is exhausted from the gap between the camera support tube 13 and the tube-like member may occur in accordance with the type of the tube-like member. Therefore, as illustrated in FIG. 13(a), it is effective that the air plug 135 for reducing leakage is provided between the tube-like member and the camera support tube 13. An example in which the tube-like member is the cannula 31 will be described below. However, this is also similar in a case where the tube-like member is the trocar or the like.

The air plug 135 in FIGS. 13(a) to 13(d) includes a holding unit 145 and a flange-like lid unit 155. The holding unit 145 has a shape of a truncated cone which is tapered (thinner toward the surface of the body) and a support tube hole 165 that penetrates the holding unit 145 is provided. The air plug 135 is configured so as to be connectable to the ground connection line 131 in the lid unit 155. In the air plug 135, a portion connected to the ground connection line 131 and a portion coming into contact with the camera support tube 13 are conductive portions. The conductive portions are integrally formed so as to electrically connect the camera support tube 13 and the ground connection line 131. The connection of the ground connection line 131 and the air plug 135 is not particularly limited as long as a method of electrically connecting the ground connection line 131 and the air plug 135 is used.

The air plug 135 is fitted to the inside of an upper end portion of the cannula 31 so as to cause the holding unit 145 to press and expand the valve 37, and is installed so as to cause the lid unit 155 to cover the top surface of the upper end portion of the cannula 31, in a state where the camera support tube 13 is held in the support tube hole 165. In this manner, the air plug 135 blocks almost of the gap between the cannula 31 and the camera support tube 13.

Since a large gap (larger gap in a case where one or two valves 37 are provided or in a case where the inner diameter of the cannula 31 is large) is generally formed between the valve 37 of the cannula 31 and the camera support tube 13, the air plug 135 is required for reducing leakage of the air. This is also similar in other modification examples which will be described below.

FIG. 13(a) illustrates an example in which the valve 37 is provided at the upper portion of the cannula 31 and is in contact with the air plug 135. However, this is just an example. Even in a case where the valve 37 and the air plug 135 are not in contact with each other, for example, a case where the valve 37 is not provided at the upper portion of the cannula 31, the air plug 135 also exhibits the effect of reducing the leakage of the air. This is also similar in other modification examples which will be described below.

FIG. 13(a) illustrates an example in which the air plug 135 is installed at the relatively large upper portion of the cannula 31. As illustrated in FIG. 13(b), even in a case (illustrated in FIG. 13(e)) where the lower portion of the holding unit 145 gradually becomes thinner, and thus the upper portion of the cannula 31 is thinner than that in the example of FIG. 13(a), the holding unit 145 can be inserted into the cannula 31 in a manner that the outer side surface of the lower portion of the holding unit 145 comes into contact with the inner side surface of the upper end portion of the cannula 31. In addition, it is possible to perform application as an air plug for various types of cannulas 31.

It is not necessary that the cannula 31 is completely sealed by the air plug 135 (completely blocks the gap between the camera support tube 13 and the cannula 31), and a gas is frequently supplied. Thus, it is sufficient that the air plug can prevent an occurrence of the leakage as much as the expanded abdominal cavity is contracted. If a little of the gap remains and the appropriate amount of the gas is leaked, an effect of lowering the temperature of the inside of the body is obtained. Thus, this case is desirable. Since the camera support tube 13 may be cooled, it is possible to improve an effect of relieving heat of the camera unit 11 to the outside via the camera support tube 13.

### SECOND MODIFICATION EXAMPLE

The air plug can function as the support tube holder that holds the camera support tube 13 so as to be fixed to the cannula 31.

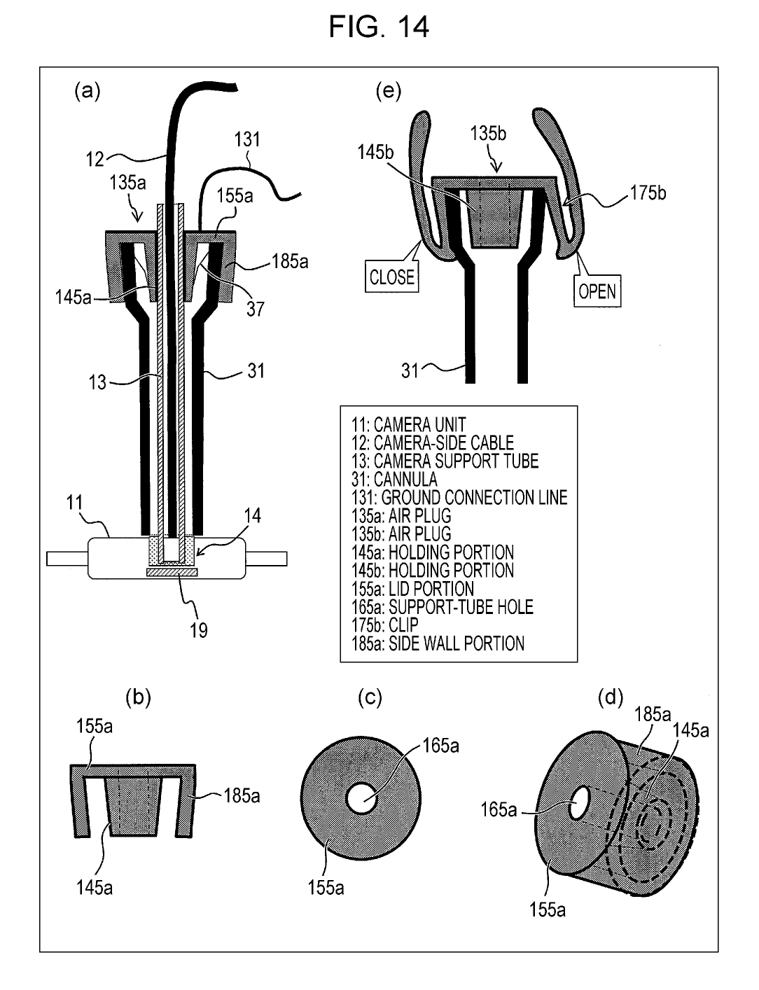

FIG. 14(a) is a sectional view illustrating the joined state of the camera support tube 13 inserted into the cannula 31 and the camera unit 11 and is a sectional view illustrating a form in which an air plug 135a having a function of the support tube holder is provided between the end portion of the camera support tube 13 on the outside of the body and the end portion of the cannula 31 on the outside of the body. FIG. 14(b) is a side view illustrating the air plug 135a. FIG. 14(c) is a top view illustrating the air plug 135a. FIG. 14(d) is a perspective view illustrating the air plug 135a. FIG. 14(e) is a sectional view illustrating another example (air plug 135b) of the air plug, in which a clip 175b which has a leaf spring shape and is used for fixing the camera support tube 13 is attached to the side surface of the air plug.

As illustrated in FIGS. 14(a) to 14(d), the air plug 135a (accessory for the support tube, support tube holder) includes a holding unit 145a, a flange-like lid unit 155a, and a side wall portion 185a. The side wall portion 185a is extended from the circumference of the lid unit 155a toward the surface of the body. The holding unit 145a has a shape of a truncated cone which is tapered (thinner toward the surface of the body) and a support tube hole 165a that penetrates the holding unit 145a is provided. Similarly to the air plug 135 described in the first modification example, the air plug 135a is configured so as to be connectable to ground connection line 131 in the lid unit 155a. In the air plug 135a, a portion connected to the ground connection line 131 and a portion coming into contact with the camera support tube 13 are conductive portions. The conductive portions are integrally formed so as to electrically connect the camera support tube 13 and the ground connection line 131. The connection of the ground connection line 131 and the air plug 135a is not particularly limited as long as a method of electrically connecting the ground connection line 131 and the air plug 135a is used.

The air plug 135a is fitted to the inside of an upper end portion of the cannula 31 so as to cause the holding unit 145a to press and expand the valve 37, and is installed so as to cause the lid unit 155a to cover the top surface of the upper end portion of the cannula 31 and to cause the side wall portion 185a to cover the outer side surface of the upper end portion of the cannula 31, in a state where the camera support tube 13 is held in the support tube hole 165a. In this manner, the air plug 135a blocks almost of the gap between the cannula 31 and the camera support tube 13 and fixes the camera support tube 13 and the cannula 31 to each other.

As illustrated in FIG. 14(e), an air plug 135b which includes a clip 175b such as leaf spring instead of the side wall portion 185a may be used. The air plug 135b has a structure of nipping the cannula 31 by opening and closing the clip 175b. With such a clip structure, the air plug 135b can be applied to various types of the cannulas (tube-like members) having different sizes, and the versatility is improved.

### THIRD MODIFICATION EXAMPLE

The air plug can function as the cable holder that holds the camera-side cable 12 so as to be fixed to the camera support tube 13.

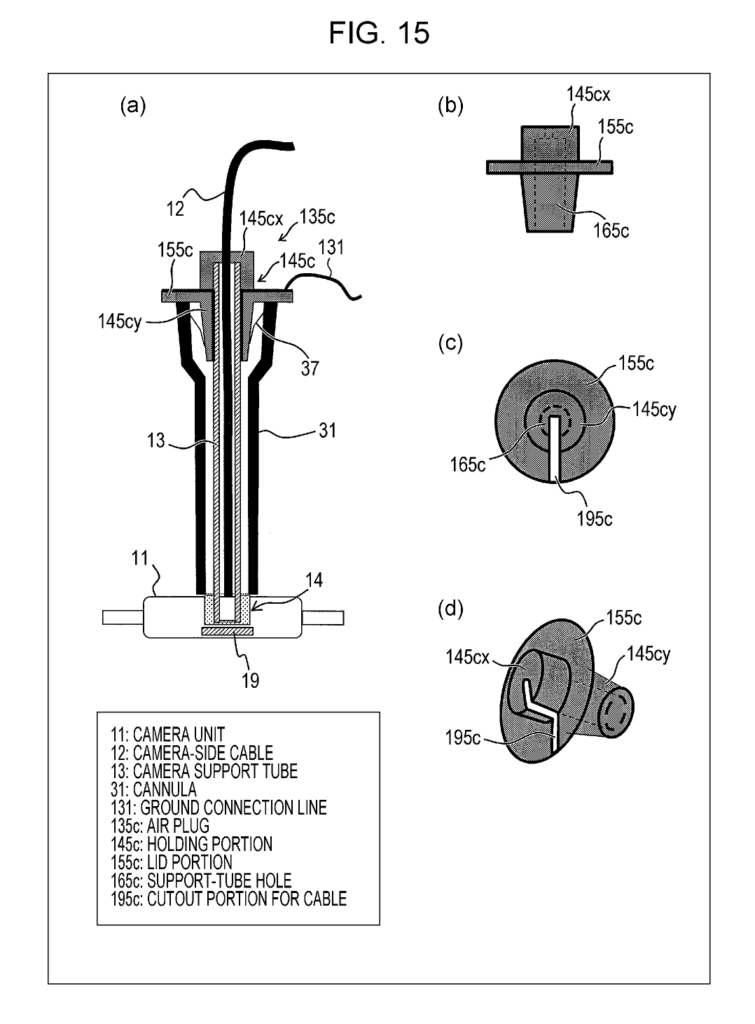

FIG. 15(a) is a sectional view illustrating the joined state of the camera support tube 13 inserted into the cannula 31 and the camera unit 11 and is a sectional view illustrating a form in which an air plug 135c having a function of the cable holder is provided between the end portion of the camera support tube 13 on the outside of the body and the end portion of the cannula 31 on the outside of the body. FIG. 15(b) is a side view illustrating an air plug 135d. FIG. 15(c) is a top view illustrating the air plug 135d. FIG. 15(d) is a perspective view illustrating the air plug 135d.

As illustrated in FIGS. 15(a) to 15(d), the air plug 135c (accessory for the support tube, cable holder) includes a holding unit 145c and a rib-like lid unit 155c. In the holding unit 145c, an upper portion 145cx positioned on an upper side of the lid unit 155c has a columnar shape and a lower portion 145cy positioned on a lower side (body surface side) of the lid unit 155c has a shape of a truncated cone which is tapered (thinner toward the surface of the body). A support tube hole 165c is provided in the holding unit 145c. A cutout 195c for the cable is provided from the center portion of the upper portion 145cx to the circumference of the lid unit 155c.

The air plug 135c is configured so as to be connectable to the ground connection line 131 in the lid unit 155c. In the air plug 135c, a portion connected to the ground connection line 131 and a portion coming into contact with the camera support tube 13 are conductive portions. The conductive portions are integrally formed so as to electrically connect the camera support tube 13 and the ground connection line 131. The connection of the ground connection line 131 and the air plug 135c is not particularly limited as long as a method of electrically connecting the ground connection line 131 and the air plug 135c is used. The air plug 135c may be configured so as to be connectable to the ground connection line 131 at the upper portion 145cx.

The air plug 135c is fitted to the inside of an upper end portion of the cannula (tube-like member) 31 so as to cause the lower portion 145cy to press and expand the valve 37, and is installed so as to cause the lid unit 155c to cover the top surface of the upper end portion of the cannula 31, in a state where the camera-side cable 12 passing through the cutout 195c for the cable is held at the center of the upper portion 145cx and the camera support tube 13 in the support tube hole 165c is held. In this manner, the air plug 135c blocks almost of the gap between the cannula 31 and the camera support tube 13 and fixes the camera support tube 13 and the camera-side cable 12 to each other.

### FOURTH MODIFICATION EXAMPLE

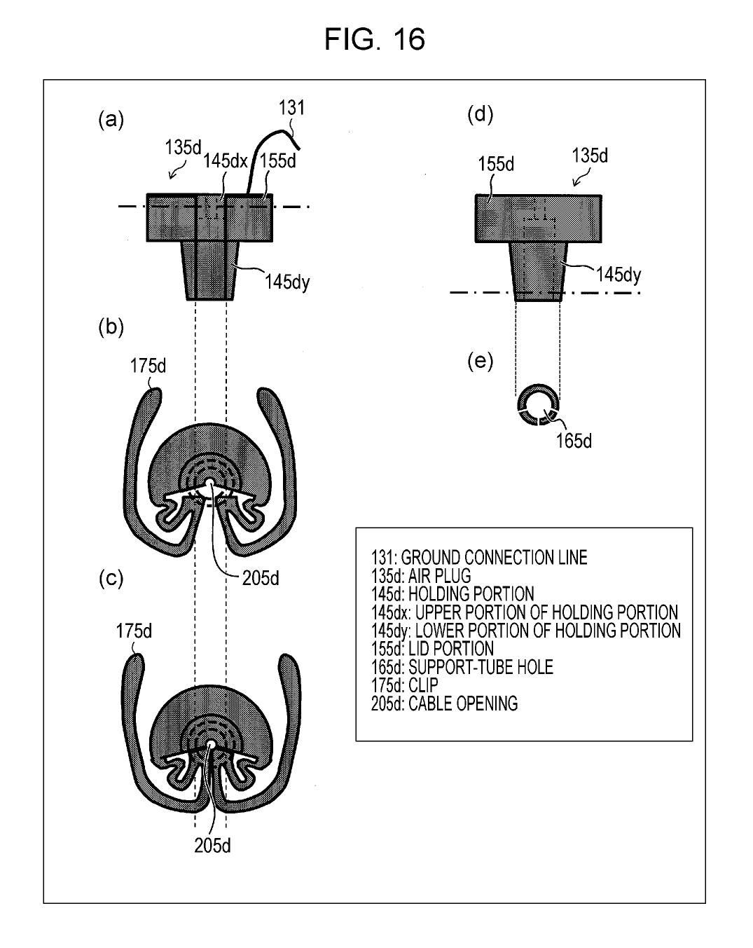

The air plug functioning as the cable holder is not limited to the air plug 135c. FIGS. 16(a) and 16(d) are longitudinal sectional views illustrating an air plug 135d which is a modification example of the air plug functioning as the cable holder. FIGS. 16(b) and 16(c) are cross-sectional view illustrating a portion taken along broken line in FIG. 16(a). FIG. 16(e) is a cross-sectional view illustrating a portion taken along broken line in FIG. 16(d). As illustrated in FIG. 16, the air plug 135d has a structure in which a clip 175d such as a leaf spring, which has a cable hole 205d is used and the camera-side cable 12 in the cable hole 205d is nipped from the side surface of the upper portion 145dx of the holding unit 145d and the lid unit 155d by opening (FIG. 16(b)) and closing (FIG. 16(c)) the clip 175d. The lower end of the holding unit 145d has a ring shape as illustrated in FIG. 16(e). With such a clip structure, it is possible to obtain a stable cable-holding force, and to apply the air plug to various types of the cables having different sizes. In addition, the versatility is improved.

As illustrated in FIG. 16(a), the air plug 135d is configured so as to be connectable to the ground connection line 131 in the lid unit 155d. In the air plug 135d, a portion connected to the ground connection line 131 and a portion coming into contact with the camera support tube 13 are conductive portions. The conductive portions are integrally formed so as to electrically connect the camera support tube 13 and the ground connection line 131. The connection of the ground connection line 131 and the air plug 135d is not particularly limited as long as a method of electrically connecting the ground connection line 131 and the air plug 135d is used.

### FIFTH MODIFICATION EXAMPLE

The air plug can function as the support tube holder that holds the camera support tube 13 so as to be fixed to the cannula 31 and as the cable holder that holds the camera-side cable 12 so as to be fixed to the camera support tube 13.

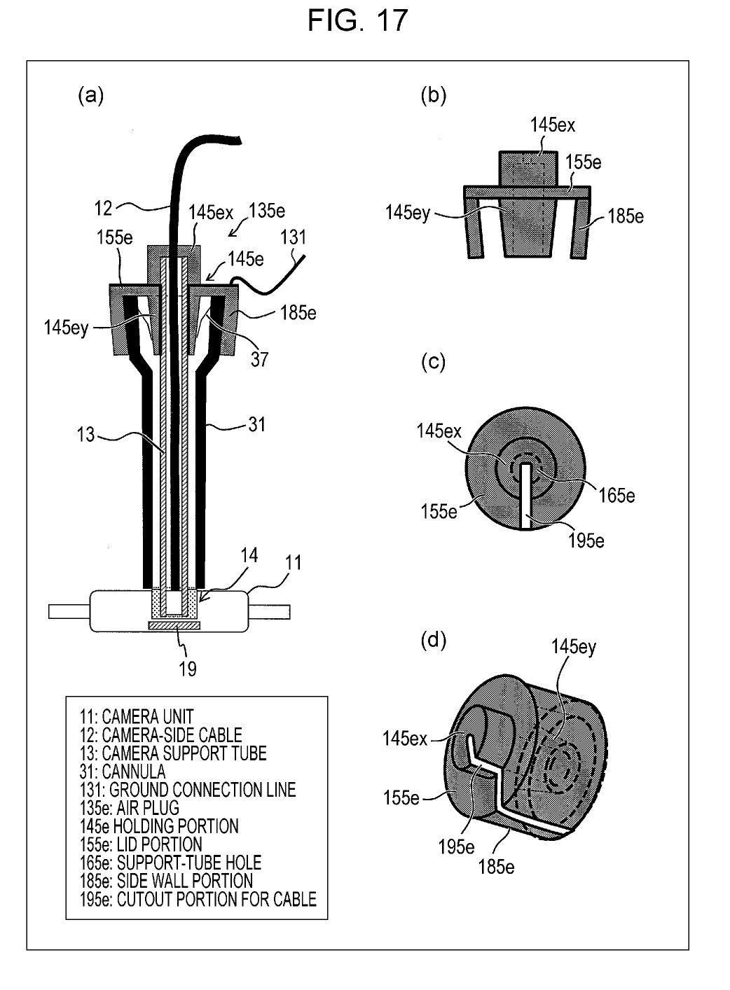

FIG. 17(a) is a sectional view illustrating the joined state of the camera support tube 13 inserted into the cannula 31 and the camera unit 11 and is a sectional view illustrating a form in which an air plug 135e having a function of the support tube holder and the cable holder is provided between the end portion of the camera support tube 13 on the outside of the body and the end portion of the cannula 31 on the outside of the body. FIG. 17(b) is a side view illustrating an air plug 135e. FIG. 17(c) is a top view illustrating the air plug 135e. FIG. 17(d) is a perspective view illustrating the air plug 135e.

As illustrated in FIGS. 17(a) to 17(d), the air plug 135e (accessory for the support tube, support tube holder, cable holder) includes a holding unit 145e, a rib-like lid unit 155e, and a side wall portion 185e. The side wall portion 185e is extended from the circumference of the lid unit 155e toward the surface of the body. In the holding unit 145e, an upper portion 145ex positioned on an upper side of the lid unit 155e has a columnar shape and a lower portion 145ey positioned on a lower side (body surface side) of the lid unit 155e has a shape of a truncated cone which is tapered (thinner toward the surface of the body). A support tube hole 165e is provided in the holding unit 145e. A cutout 195e for the cable is provided from the center portion of the upper portion 145ex to the lower end of the side wall portion 185e via the circumference of the lid unit 155e.

The air plug 135e is configured so as to be connectable to the ground connection line 131 in the lid unit 155e. In the air plug 135e, a portion connected to the ground connection line 131 and a portion coming into contact with the camera support tube 13 are conductive portions. The conductive portions are integrally formed so as to electrically connect the camera support tube 13 and the ground connection line 131. The connection of the ground connection line 131 and the air plug 135e is not particularly limited as long as a method of electrically connecting the ground connection line 131 and the air plug 135e is used. The air plug 135e may be configured so as to be connectable to the ground connection line 131 at the upper portion 145ex.

The air plug 135e is fitted to the inside of an upper end portion of the cannula (tube-like member) 31 so as to cause the lower portion 145ey to press and expand the valve 37, and is installed so as to cause the lid unit 155e to cover the top surface of the upper end portion of the cannula 31 and to cause the side wall portion 185e to cover the outer side surface of the upper end portion of the cannula 31, in a state where the camera-side cable 12 passing through the cutout 195e for the cable is held at the center of the upper portion 145ex and the camera support tube 13 in the support tube hole 165e is held. In this manner, the air plug 135e blocks almost of the gap between the cannula 31 and the camera support tube 13 and fixes the camera support tube 13 and the camera-side cable 12 to each other.

### SIXTH MODIFICATION EXAMPLE

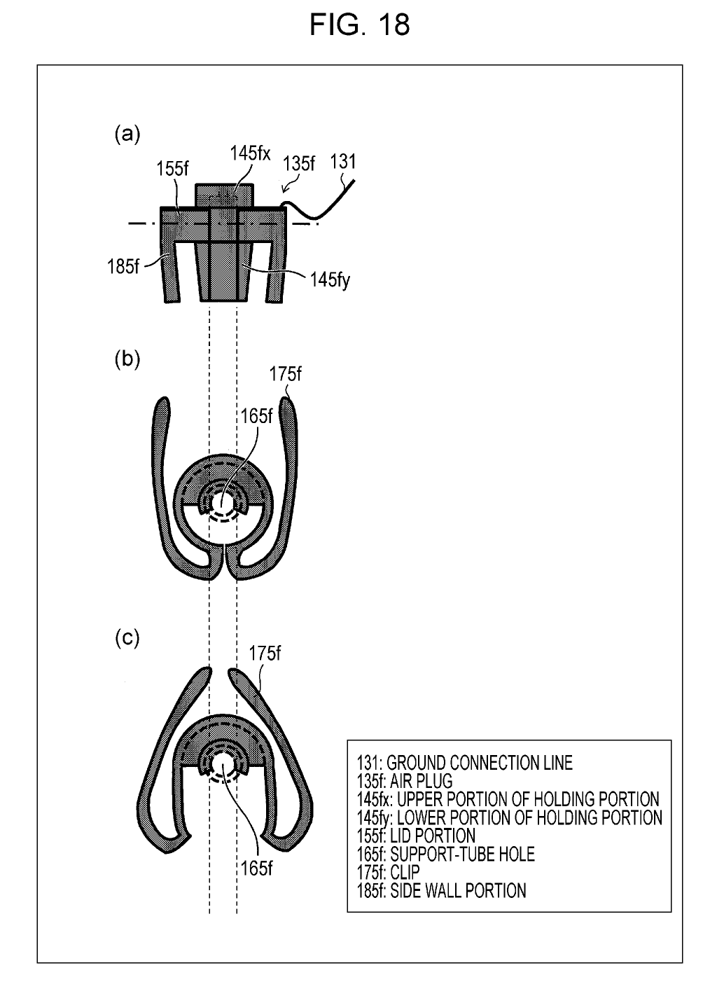

The air plug which functions as the cable holder and the support tube holder is not limited to the air plug 135e. FIG. 18(a) is a longitudinal sectional view illustrating an air plug 135f which is a modification example of the air plug functioning as the cable holder and the support tube holder. FIGS. 18(b) and 18(c) are cross-sectional views illustrating a portion taken along broken line in FIG. 18(a). As illustrated in FIGS. 18(a) to 18(c), the air plug 135f has a structure, at the upper portion 145fx of a holding unit 145f and a lid unit 155f of the air plug 135f, in which a clip 175f such as a leaf spring, which has a support tube hole 165f is used and the camera support tube 13 in the support tube hole 165f is nipped from the side surface thereof by opening and closing the clip 175f. With such a clip structure, it is possible to obtain a stable holding force.

As illustrated in FIG. 18(a), the air plug 135f is configured so as to be connectable to the ground connection line 131 in the lid unit 155f. In the air plug 135f, a portion connected to the ground connection line 131 and a portion coming into contact with the camera support tube 13 are conductive portions. The conductive portions are integrally formed so as to electrically connect the camera support tube 13 and the ground connection line 131. The connection of the ground connection line 131 and the air plug 135f is not particularly limited as long as a method of electrically connecting the ground connection line 131 and the air plug 135f is used. The air plug 135f may be configured so as to be connectable to the ground connection line 131 at the upper portion 145fx.

The air plug according to the modification example may have a structure in which a clip such as a leaf spring, which has a cable hole is used, and the camera-side cable 12 in the cable hole is nipped from the side surface of the upper portion of the holding unit by opening and closing the clip. With such a clip structure, it is possible to obtain a stable cable-holding force, and to apply the air plug to various types of the camera-side cables having different sizes. In addition, the versatility is improved.

The air plug according to the modification example may have a structure in which a cable holder configured by a clip such as a leaf spring, which has a cable hole is provided at the upper portion of the holding unit, the lower end of the holding unit is set to have a ring shape, and a clip such as a leaf spring, which fixes the camera support tube 13 is provided between the cable holder and the lower end of the holding unit. With such a double clip structure, it is possible to obtain a stable holding force of the camera support tube 13 and the camera-side cable 12, and to apply the air plug to various types of the camera-side cables, the camera support tubes, and the cannulas (tube-like member) having different sizes. In addition, the versatility is improved.

### Embodiment 4

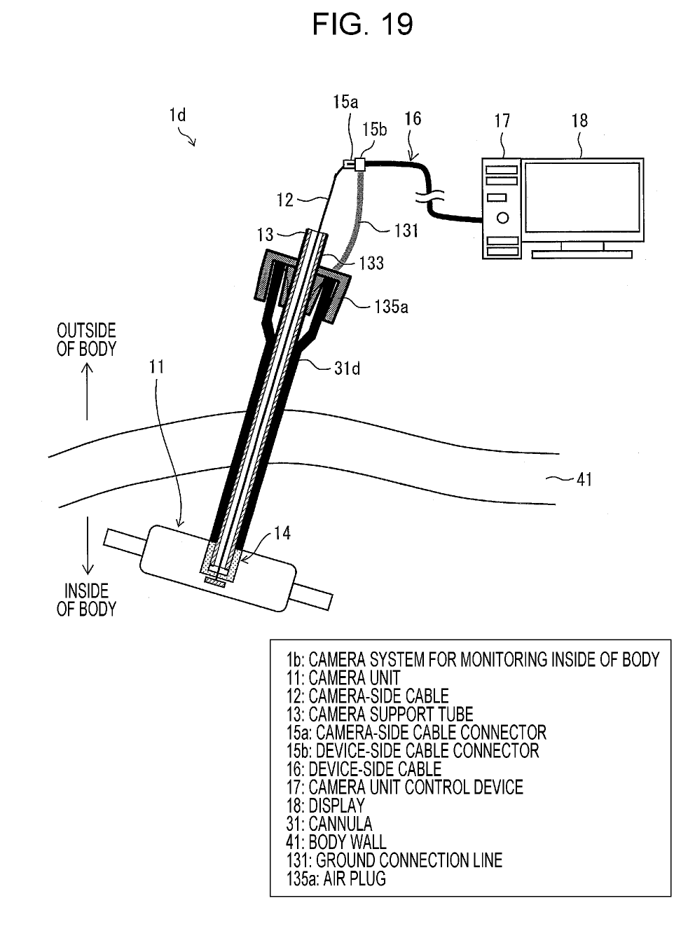

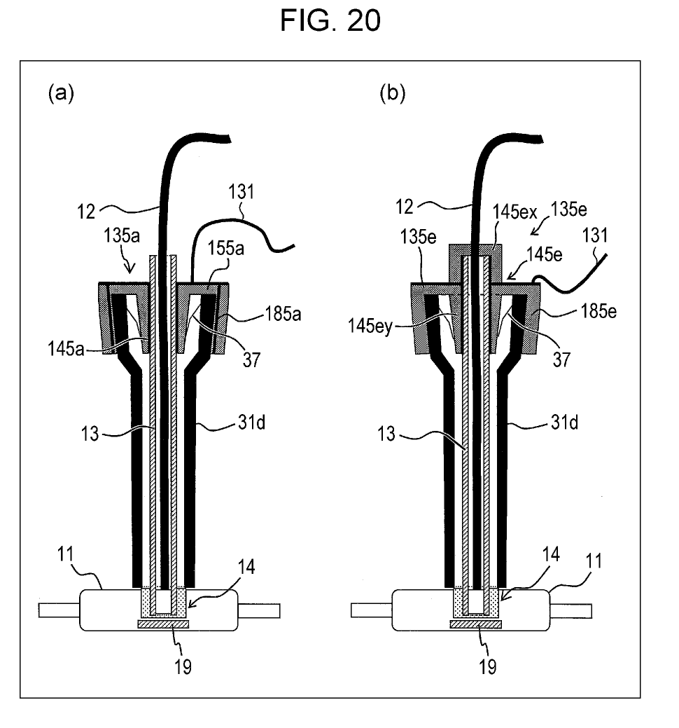

Still another embodiment of the present invention will be described based on FIGS. 19 and 20 as follows. For easy description, members having the same function as those described in the above embodiment are denoted by the same reference signs and descriptions thereof will not be repeated.

In the embodiment, a configuration in which the tube-like member (cannula 31d in the embodiment) is formed of a conductive material, and thus a triple electrical shield is realized will be described. FIG. 19 is a schematic diagram illustrating a configuration of a camera system 1d for monitoring an inside of a body, according to this embodiment. Similarly to Embodiment 3, in FIG. 19, the support-tube handle unit 132 is omitted. However, the camera support tube 13 may include the support-tube handle unit 132.

A camera system 1d for monitoring an inside of a body, according to the embodiment includes the cannula 31d formed of a conductive material. The camera support tube 13 is fixed to the cannula 31 by the air plug 135a (second modification example of Embodiment 3, see FIG. 14) which has a function of the support tube holder. As illustrated in FIG. 19, the air plug 135a is electrically connected to the grounded device-side cable connector 15b via the ground connection line 131. Thus, it is possible to cause the high-frequency noise to flow from the camera support tube 13 and the cannula 31d into the ground via the ground connection line 131 and the device-side cable connector 15b. Accordingly, since a triple electrical shield is obtained, it is possible to more reduce mixing of the high-frequency noise into an image signal. Since the tube-like member such as the cannula, which is generally used in the camera system for monitoring an inside of a body is used as the electrical shield, it is possible to maintain minimal invasiveness and to improve the function of a shield against the high-frequency noise.

The configuration of the air plug according to the embodiment will be described in detail with reference to FIG. 20. FIG. 20(a) is a sectional view illustrating the joined state of the camera support tube 13 inserted into the cannula 31d and the camera unit 11 and is a sectional view illustrating a form in which the air plug 135a is provided between the end portion of the camera support tube 13 on the outside of the body and the end portion of the cannula 31d on the outside of the body.

The air plug 135a according to the embodiment is different from the air plug 135a described in the second modification example of Embodiment 3. In this air plug 135a, a portion coming into contact with the cannula 31d (surface of the side wall portion 185a, which is in contact with the cannula 31d) is a conductive portion. The conductive portion is integrally formed so as to electrically connect the camera support tube 13 and the cannula 31d with the ground connection line 131. Thus, it is possible to cause the high-frequency noise from the camera support tube 13 and the cannula 31d to flow into the ground via the ground connection line 131 and the device-side cable connector 15b.

The air plug used in the embodiment may come into contact with the camera support tube 13 and the cannula 31d, and is not limited to the air plug 135a. For example, the air plug 135e described in the fifth modification example of Embodiment 3 may be used. FIG. 20(b) is a sectional view illustrating the joined state of the camera support tube 13 inserted into the cannula 31 and the camera unit 11, and is a sectional view illustrating a form in which the air plug 135e is provided between the end portion of the camera support tube 13 on the outside of the body and the end portion of the cannula 31d on the outside of the body.

The air plug 135e according to the embodiment is different from the air plug 135e described in the fifth modification example of Embodiment 3. In this air plug 135e, a portion coming into contact with the cannula 31d (surface of the side wall portion 185e, which is in contact with the cannula 31d) is a conductive portion. The conductive portion is integrally formed so as to electrically connect the camera support tube 13 and the cannula 31d with the ground connection line 131.

In a case where the tube-like member such as a cannula is formed of a conductive material, safety for the body is improved. Thus, it is preferable that the surface of the tube-like member is coated with a non-conductive material such as resin. In the embodiment, an example in which the cannula is used as the tube-like member, and the tube-like member may be a member other than the cannula such as a trocar.

### Embodiment 5

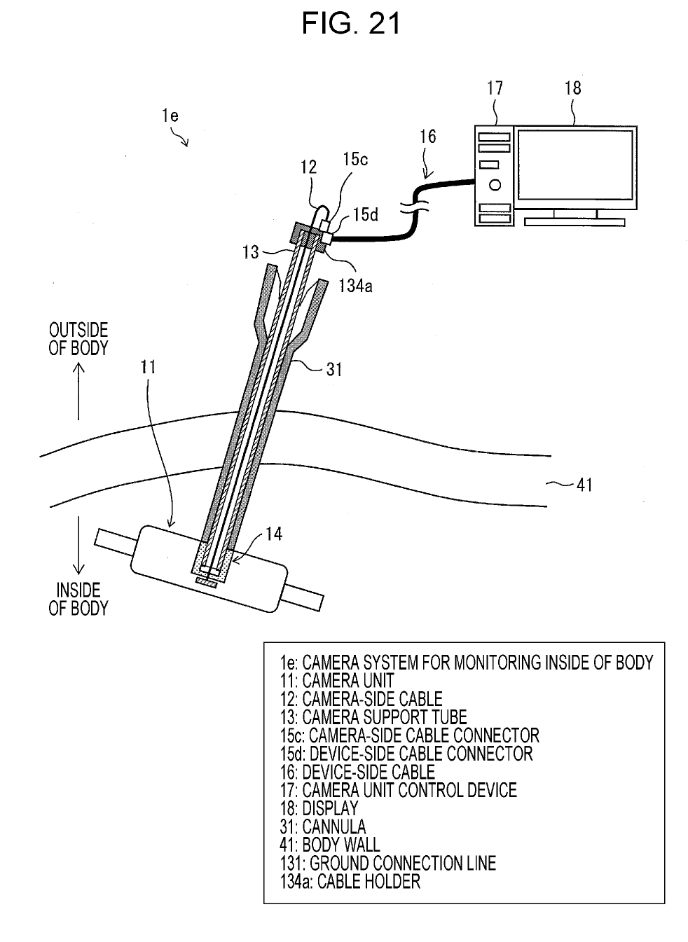

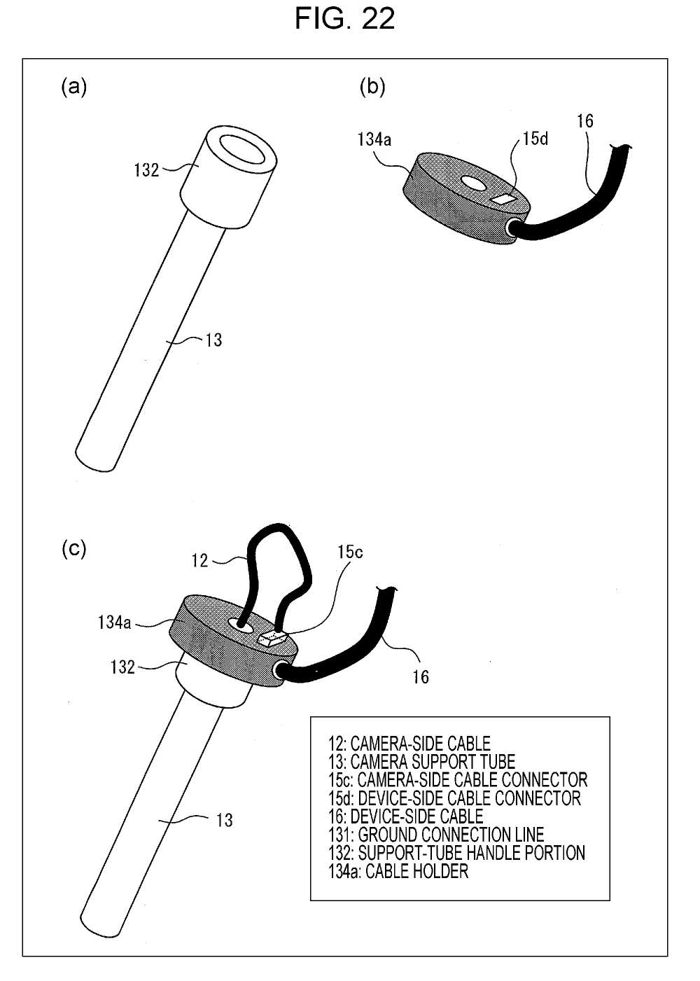

Still another embodiment of the present invention will be described based on FIGS. 21 and 22 as follows. For easy description, members having the same function as those described in the above embodiment are denoted by the same reference signs and descriptions thereof will not be repeated.

In the above-described Embodiments 2 to 4, the configuration in which the conductive camera support tube 13 or the cannula 31 is installed by using the ground connection line 131 is described. In the embodiment, a configuration in which the camera support tube 13 and the grounded device-side cable connector 15b are electrically connected to each other without passing through the ground connection line 131 will be described. FIG. 21 is a schematic diagram illustrating a configuration of a camera system 1e for monitoring an inside of a body, according to this embodiment. Similarly to Embodiments 2 to 4, in FIG. 21, for simple illustrations, the support-tube handle unit 132 is omitted.

A camera system 1e for monitoring an inside of a body, according to the embodiment includes a cable holder 134a which is attached to the camera support tube 13 so as to fix the camera-side cable 12 to the camera support tube 13. Here, details of the camera support tube 13 and the cable holder 134a according to the embodiment will be described in detail with reference to FIG. 22. FIG. 22(a) is a perspective view illustrating the camera support tube 13 according to this embodiment. FIG. 22(b) is a perspective view illustrating a cable holder 134a according to this embodiment, and FIG. 22(c) is a perspective view illustrating a joined state of the camera support tube 13 and the cable holder 134a according to this embodiment.

As illustrated in FIG. 22(a), the camera support tube 13 according to the embodiment includes the support-tube handle unit 132. As illustrated in FIG. 22(b), regarding the cable holder 134a according to the embodiment, the device-side cable 16 is fixed and the device-side cable connector 15d is built (integrated). As illustrated in FIG. 22(c), a camera-side cable connector 15c is connected to the device-side cable connector 15d provided in the cable holder 134a, and thus the camera unit 11 and the camera unit control device 17 are connected to each other.

In the cable holder 134a, a portion coming into contact with the camera support tube 13 is a conductive portion and the conductive portion is electrically connected to the device-side cable connector 15d. Thus, it is possible to cause the high-frequency noise to flow from the camera support tube 13 into the ground via the grounded device-side cable connector 15d. It is not necessary for using the ground connection line. Thus, it is possible to simplify the configuration of the camera system for monitoring an inside of a body. It is possible to prevent the disadvantage of the ground connection line interfering with the operation of the camera unit 11.

The configuration in the embodiment can be also applied to the above-described Embodiments 3 and 4. Specifically, the accessory for the support tube (cable holder, support tube holder, and air plug) described in Embodiments 3 and 4 may be integrated with the device-side cable connector 15d.

### Embodiment 6

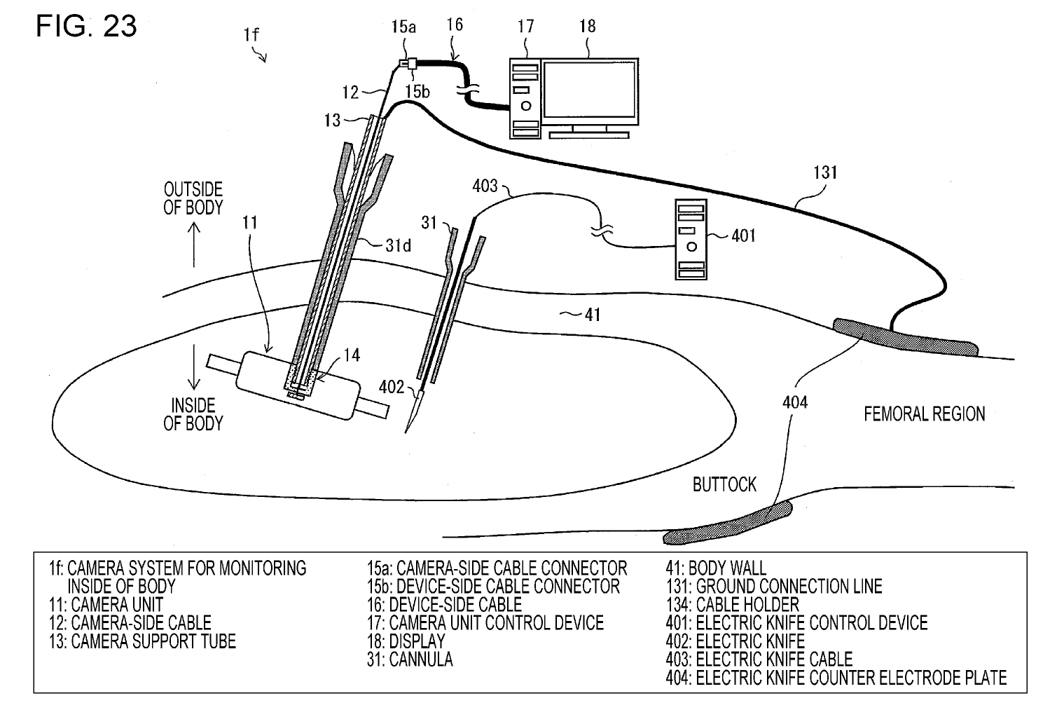

Still another embodiment of the present invention will be described based on FIG. 23 as follows. For easy description, members having the same function as those described in the above embodiment are denoted by the same reference signs and descriptions thereof will not be repeated.

FIG. 23 is a schematic diagram illustrating a configuration of a camera system if for monitoring an inside of a body, according to this embodiment. As illustrated in FIG. 23, a camera system if for monitoring an inside of a body, according to the embodiment has a configuration in which the camera support tube 13 and is electrically connected with an electric-knife counter electrode plate (counter electrode plate) 404 which is grounded, via the ground connection line 131. Thus, since the camera support tube 13 is grounded, it is possible to cause the high-frequency noise to flow from the camera support tube 13 into the ground.

Regarding the connection of the camera support tube 13 and the ground connection line 131, the method is not particularly limited as long as the ground connection line 131 and the camera support tube 13 can be electrically connected to each other. For example, the connection method described in the above-described Embodiments 2 to 5 can be applied. Regarding a method of connecting the ground connection line 131 and the electric-knife counter electrode plate 404, the method is not particularly limited as long as both of the ground connection line 131 and the electric-knife counter electrode plate 404 can be electrically connected to each other. For example, a connector (not illustrated) that fixes the lead wire of the ground connection line 131 may be provided on the electric-knife counter electrode plate 404 and the ground connection line 131 may be fixed to the electric-knife counter electrode plate 404 in a state where detachment is not possible.

The camera support tube 13 may be electrically connected to a current feedback terminal (not illustrated) of an electric knife 402 via the ground connection line 131.

### Common Modification Example in Embodiments 1 to 6

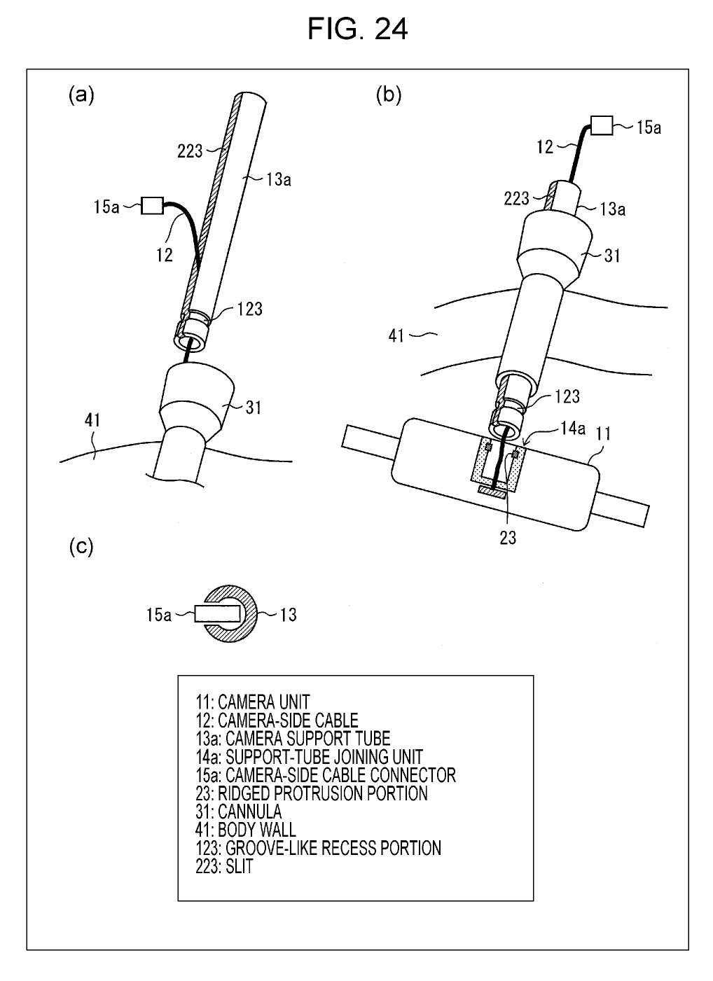

FIG. 24 is a diagram illustrating a modification example of the camera support tube and the support-tube joining unit. As illustrated in FIGS. 24(a) to 24(c), a slit 223 is provided on the side surface of the camera support tube 13a. The slit 223 is set to have a width which is sufficiently smaller than the wavelength of a high frequency wave, in order to hold the function as the electrical shield. The slit 223 illustrated in FIGS. 24(a) to 24(c) has a width which is larger than the actual width in order to make the drawings easy to understand. The slit 223 is provided, and thus an installation method as follows can be applied in a case where the camera system 1 for monitoring an inside of a body is installed.

As illustrated in FIG. 24(a), firstly, the camera-side cable 12 is pulled up from the cannula 31, and then the portion of the camera-side cable 12 is inserted into the lower portion of the slit 223 of the camera support tube 13a.

Then, the camera support tube 13a is inserted into the cannula 31 so as to cause the slit 223 to follow the camera-side cable 12. That is, as illustrated in FIG. 24(b), the camera-side cable 12 is pulled upwardly in a state where the portion of the camera-side cable 12 is inserted into the lower portion of the slit 223 of the camera support tube 13a, and at the same time, the camera support tube 13a is inserted into the cannula 31 by using the camera-side cable 12 as the guide.

As described above, the slit 223 is provided on the side surface of the camera support tube 13a, and thus the camera-side cable 12 can be inserted into the inside of the camera support tube 13a from the slit 223 when the camera-side cable 12 passes through the inside of the camera support tube 13.

Thus, it is possible to easily cause the camera-side cable 12 to pass through the inside of the camera support tube 13 and a work of inserting the camera support tube 13a into the inside of the body is simplified.

In a case where the cannula 31 is used for guiding the camera support tube 13a toward the inside of the body and in a case where the slit 223 is not provided, the length obtained by adding the lengths of at least the cannula 31 and the camera support tube 13a is required for causing the camera-side cable 12 which has been drawn out from the cannula 31 to pass through the inside of the camera support tube 13a.

However, in a case where the slit 223 is provided, the length of the camera-side cable 12 may be longer than the length of the cannula 31. Accordingly, as described above, the slit 223 is provided in the camera support tube 13a, and thus it is possible to reduce the limitation in the length of the camera-side cable 12.

As illustrated in FIG. 24(c), the slit 223 is provided in the camera support tube 13a, and thus external dimensions of the camera-side cable connector 15a connected to the camera-side cable 12 can be set to be greater than the inner diameter of the camera support tube 13. Accordingly, the camera-side cable connector 15a is easily connected to the device-side cable 16, and thus it is possible to increase work efficiency. The external dimensions of the camera-side cable connector 15a are set to be smaller than the inner diameter of the cannula 31.

In the embodiment, a configuration in which the ridged protrusion portion 23 of the support-tube joining unit 14a is fitted to the groove-like recess portion of the camera support tube 13a in which the slit 223 is provided is described. However, the camera support tube 13a is not limited to the configuration of including the groove-like recess portion 123. In the support-tube joining unit 14a, the structure of a portion joined to the camera support tube 13a is changed depending on the structure of the camera support tube 13a.

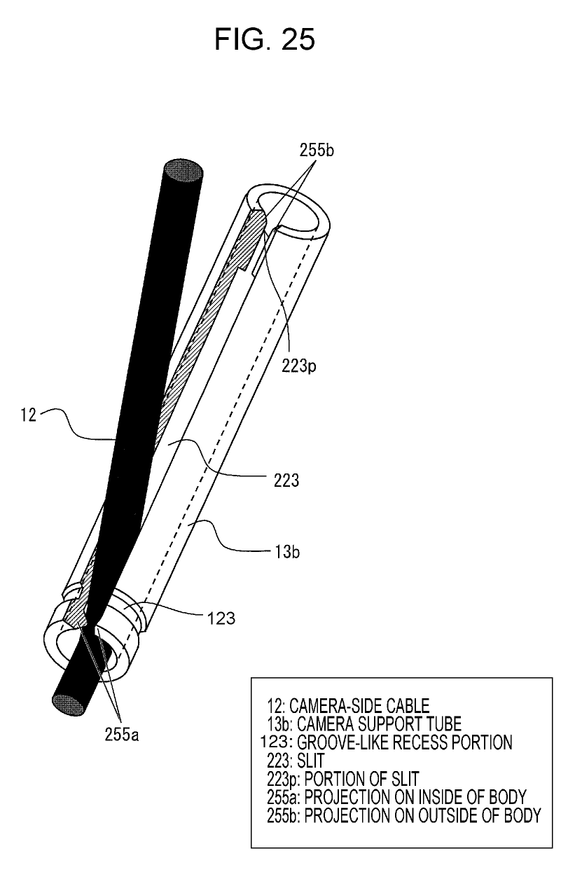

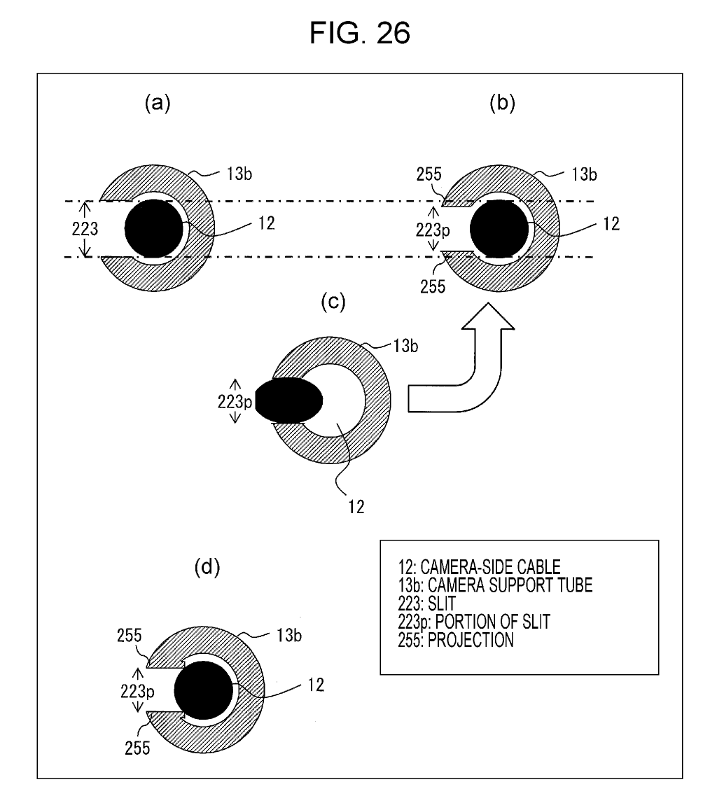

The camera support tube including the slit 223 may have a configuration as illustrated in FIGS. 25 and 26. FIG. 25 is a diagram illustrating another modification example of the camera support tube according to Embodiment 1 to 6. FIG. 26(a) is a sectional view illustrating the camera support tube illustrated in FIG. 24. FIGS. 26(b) and 26(c) are sectional views illustrating the camera support tube illustrated in FIG. 25. FIG. 26(d) is a sectional view illustrating still another modification example of the camera support tube.

As illustrated in FIG. 25, both ends of the camera support tube 13b in the modification example form pair projections 255 at both edges of the slit 223, so as to face each other.

Thus, as illustrated in FIG. 26(a), in the above-described camera support tube 13a, the width of the slit 223 is greater than the diameter of the camera-side cable 12. However, in the camera support tube 13b, as illustrated in FIG. 26(b), a portion 223p of the slit, which corresponds to the pair projection 255, is smaller than the diameter of the camera-side cable 12. Accordingly, when the camera-side cable 12 passes through the camera support tube 13b, the camera-side cable 12 can temporarily elastically deformed at the portion 223p of the slit, so as to pass (see FIG. 26(c)). After passing, the diameter of the camera-side cable 12 is originally brought back (see FIG. 26(b)). Thus, the camera-side cable 12 is not easily removed from the camera support tube 13b, and the workability is gradually improved when the camera support tube 13 is installed.

As illustrated in FIG. 25, at this time, it is preferable that the projection 255b on the outside of the body, which is provided at the body-outside end portion of the camera support tube 13b has a length (simply referred to as “the length” below) in a height direction of the camera support tube 13b, which is longer than the projection 255a on the inside of the body, which is provided at the body-inside end portion of the camera support tube 13b.

As described above, in the camera support tube having the slit 223, the portion of the camera-side cable 12 is inserted into the lower portion (that is, end portion on the inside of the body) of the slit 223 of the camera support tube 13a. Therefore, the projection 255a on the inside of the body has a reduced length so as not to easily insert the camera-side cable 12. The projection 255b on the outside of the body has the increased length so as to firmly fix the camera-side cable 12 to the camera support tube 13b.

As illustrated in FIG. 26(d), instead of increasing the length thereof, the projection 255b on the outside of the body may have a projection which is extended in an internal direction of the camera support tube 13b, as illustrated in FIG. 26(d). Thus, it is possible to fix the camera-side cable 12 by the projection extended in the internal direction. The projection 255b on the outside of the body may have the increased length and have the projection extended in the internal direction.

The camera support tube 13a and the camera support tube 13b described in the modification example can be also applied to a camera system for monitoring an inside of a body, which does not have an electrical shield function.

### [Overview]

According to Aspect 1 of the present invention, a camera system for monitoring an inside of a body (1) includes a support tube (camera support tube 13) which has one end portion guided toward an inside of a body and is formed of a conductive material, a ground unit (metal spring 191) which has at least a portion formed of a conductive material and causes the support tube to be grounded, an imaging unit (camera unit 11) which is joined to the support tube inside the body, a joining unit (support-tube joining unit 14 and heat-conductive protrusion portion 14d) that joins the imaging unit and the support tube, a first cable (camera-side cable 12) which is connected to the imaging unit and is drawn toward an outside of the body through the support tube, and a control system (3) which is provided on the outside of the body, electrically connected to the first cable, and includes at least a display device (display 18).

According to the above configuration, since the support tube is formed of a conductive material and the support tube is grounded, it is possible to cause high-frequency noise (noise current) generated from the electric knife and the like to flow from the support tube into the ground. Thus, it is possible to prevent mixing of a high-frequency noise into a signal passing in the first cable. Accordingly, it is possible to reduce an influence of the high-frequency noise (it is possible to realize the support tube having the electrical shield function).

Since the support tube and the imaging unit are joined to each other and the first cable is guided toward the outside of the body through the inside of the support tube, a situation in which a load is applied to the first cable, or the first cable is exposed in the body or is brought into contact with the body wall does not occur. Thus, reliability (water repellency and antifouling property of the connection portion) of the first cable is improved. From the above descriptions, it is possible to realize the camera system for monitoring an inside of a body, which has high reliability.

The surgeon can operate the support tube in accordance with the situation, so as to change the direction (direction of the visual field) of the imaging unit. Specifically, it is possible to change the direction of the imaging unit by using an elastic force of the body wall and by inclining the support tube. At this time, if the surgeon separates the hand from the support tube, the direction thereof is brought back to the original direction by the elastic force of the body wall. Thus, it is possible to improve work efficiency of the surgeon. If the support tube is set to be a cylindrical tube, it is possible to easily rotate the support tube in a circumferential direction. Thus, the surgeon can change the direction of the imaging unit without applying a load on the body wall. If the support tube is held by the tube-like member such as the cannula, so as to be movable in a longitudinal direction (extension direction of the tube), the surgeon pushes the support tube to the inside of the body or pulls the support tube to the outside of the body, and thus can change an imaging zoom without applying a load to the body wall. From the above descriptions, it is possible to realize the camera system for monitoring an inside of a body, which has ease of use.

According to Aspect 2 of the present invention, in Aspect 1, in the camera system for monitoring an inside of a body, the support tube may have at least a portion which is non-conductive and is capable of being brought into contact with a body wall when being joined to the imaging unit.

According to the above configuration, since the portion which is capable of being brought into contact with the counter wall when being joined to the imaging unit is non-conductive, in a case where the support tube is used in a state of directly coming into contact with the body wall, there is no influence of the high-frequency noise (noise current) flowing in the support tube on the body. Accordingly, it is possible to improve safety of the body.

Since the portion of the joining unit, which is joined to the imaging unit, is not non-conductive, it is possible to maintain heat dissipation properties of heat or conductivity of a current from the imaging unit via the joining unit.

According to Aspect 3 of the present invention, in Aspect 1 or 2, in the camera system for monitoring an inside of a body, a portion of the joining unit, which is brought into contact with the support tube may have at least a part which is formed of a conductive material, and the ground unit may be electrically connected to the support tube, the joining unit, and a grounded circuit board of the imaging unit.

According to the above configuration, since the grounded circuit board is electrically connected to the support tube, the joining unit, and the imaging unit, it is possible to cause high-frequency noise (noise current) generated from the electric knife and the like to flow from the support tube into the ground. Since the support tube, the ground unit, and the portion of the joining unit, which is formed of a conductive material are in contact with each other, the portion thereof can transfer heat from the circuit board to the support tube and dissipate the heat to the outside. Accordingly, it is possible to realize a camera system for monitoring an inside of a body, which has the electrical shield function and a heat-transferring (heat dissipation) function together.

According to Aspect 4 of the present invention, in Aspect 1 or 2, the camera system for monitoring an inside of a body may further include a second cable which is electrically connected to the control system. The first connector provided on the first cable may be fitted to a second connector provided on the second cable, and the ground unit may be electrically connected to any one of the first connector and the second connector which is grounded.

According to the above configuration, since the ground line which is connectable to the support tube is connected to the first connector or the second connector which is grounded, it is possible to cause high-frequency noise (noise current) generated from the electric knife and the like to flow from the support tube into the ground. Accordingly, it is possible to realize a support tube having the electrical shield function.

According to Aspect 5 of the present invention, in Aspect 4, in the camera system for monitoring an inside of a body, the ground unit may be connected to the grounded second connector.

Since the first connector is provided on the first cable, if the ground line is connected to the first connector, interference with the work may occur. Here, according to the above configuration, since the ground line is connected to the second connector, it is possible to prevent an occurrence in which the ground line interferes with the work of the surgeon.

According to Aspect 6 of the present invention, in Aspect 4 or 5, in the camera system for monitoring an inside of a body, the ground unit may be a ground line which is capable of being electrically connected to the support tube.

According to the above configuration, the ground line is connected to the support tube, and thus the support tube is grounded. Thus, it is possible to cause the support tube to be grounded by a simple method and to realize the electrical shield.

According to Aspect 7 of the present invention, in Aspect 6, in the camera system for monitoring an inside of a body, the ground line may be detachable from the support tube through a ground-line connector or a support-tube connection line which is provided on the support tube. The support-tube connection line may have such a length that it does not come into contact with a cannula when the support tube is inserted into the cannula having an annular structure in which the support tube is capable of being inserted in the cannula.

According to the above configuration, since the ground line and the support tube are connected so as to be detachable from each other, a situation in which the ground line is in the way, for example, when the support tube is collected does not occur. Since the support-tube connection line has such a length that it does not come into contact with the cannula, it is possible to prevent an occurrence in which the support-tube connection line interferes with the work of a surgeon.

According to Aspect 8 of the present invention, in Aspect 6, the camera system for monitoring an inside of a body may further include an accessory for the support tube, which is connectable to the ground line and is attachable to the support tube or a body-outside end portion of the cannula having an annular structure in which the support tube is capable of being inserted in the cannula, so as to be brought into contact with the support tube. The accessory for the support tube may have at least a portion which is formed of a conductive material so as to electrically connect the support tube to the ground line.

According to the above configuration, the accessory for the support tube has at least the portion which is formed of a conductive material so as to electrically connect the support tube to the ground line. Thus, it is possible to cause the high-frequency noise from the support tube to flow into the ground via the accessory for the support tube. Since the accessory for the support tube is connectable to the ground line, it is possible to realize a support tube having the electrical shield function even if the support tube which is connectable to the ground line is not prepared.

The sentence of “at least the portion being formed of a conductive material so as to electrically connect the support tube to the ground line” means, for example, that a portion coming into contact with the support tube and a portion connected to the ground connection line are formed of a conductive material and the two portions are integrally configured. Examples of the specific example of the accessory for the support tube include the cable holder that fixes the first cable to the support tube, the support tube holder that fixes the support tube to the cannula, and the air plug that blocks at least a portion of a gap between the support tube and the cannula.

According to Aspect 9 of the present invention, in Aspect 6, in the camera system for monitoring an inside of a body, the ground line may be electrically connected to a grounded current feedback terminal of an electric knife or a counter electrode plate attached to a patient.

According to the above configuration, since the ground line which is connectable to the support tube is connected to the current feedback terminal or the counter electrode plate of the electric knife, which is grounded, it is possible to cause high-frequency noise (noise current) generated from the electric knife and the like to flow from the support tube into the ground. Accordingly, it is possible to realize a support tube having the electrical shield function.

According to Aspect 10 of the present invention, in any of Aspects 1 to 9, in the camera system for monitoring an inside of a body, a slit may be formed in the support tube so as to enable the first cable to pass from the side surface of the support tube to the inside of the support tube.

According to the above configuration, since the slit is formed on the side surface of the support tube, it is possible to easily cause the first cable to pass to the inside of the support tube. In addition, a work of inserting the support tube into the inside of the body is simplified.

If the width of the slit is set to be smaller than the wavelength of a high-frequency noise, it is possible to hold the electrical shield function of the support tube.

According to Aspect 11 of the present invention, in Aspect 10, in the camera system for monitoring an inside of a body, an edge of the slit may include a portion forming a projection, and a portion of the slit, which corresponds to the portion forming the projection may have a width narrower than the diameter of the first cable.

According to the above configuration, since the width of a portion of the slit is smaller than the diameter of the cable by the portion forming the projection, the camera-side cable is not easily removed from the camera support tube, and the workability is gradually improved when the camera support tube is installed.

According to Aspect 12 of the present invention, in any of Aspects 1 to 11, in the camera system for monitoring an inside of a body, the support tube may have a gripping unit (support-tube handle unit 132) at a body-outside end portion. The gripping unit has an outer diameter greater than an inner diameter of the cannula having an annular structure in which the support tube is capable of being inserted in the cannula.

According to the above configuration, since the outer diameter of the gripping unit is greater than the inner diameter of the cannula, a situation in which the support tube passes through the cannula does not occur. Accordingly, it is possible to prevent falling of the support tube to the inside of the body.

According to Aspect 13 of the present invention, a camera system for monitoring an inside of a body includes a support tube which has one end portion guided toward an inside of a body, an imaging unit which is joined to the support tube inside the body, a joining unit that joins the imaging unit and the support tube, a first cable which is connected to the imaging unit and is drawn toward an outside of the body through the support tube, and a control system which is provided on the outside of the body, electrically connected to the first cable, and includes at least a display device. The support tube has a gripping unit at a body-outside end portion, and the gripping unit has an outer diameter greater than an inner diameter of the cannula having an annular structure in which the support tube is capable of being inserted in the cannula.

According to the above configuration, since the outer diameter of the gripping unit is greater than the inner diameter of the cannula, a situation in which the support tube passes through the cannula does not occur. Accordingly, it is possible to prevent falling of the support tube to the inside of the body.

According to Aspect 14 of the present invention, a camera system for monitoring an inside of a body includes a support tube which has one end portion guided toward an inside of a body, an imaging unit which is joined to the support tube inside the body, a joining unit that joins the imaging unit and the support tube, a first cable which is connected to the imaging unit and is drawn toward an outside of the body through the support tube, and a control system which is provided on the outside of the body, electrically connected to the first cable, and includes at least a display device. A slit is formed in the support tube so as to enable the first cable to pass from a side surface of the support tube to inside of the support tube. An edge of the slit includes a portion forming a projection, and a portion of the slit, which corresponds to the portion forming the projection, has a width narrower than a diameter of the first cable.

According to the above configuration, since the slit is formed on the side surface of the support tube, it is possible to easily cause the first cable to pass to the inside of the support tube. In addition, a work of inserting the support tube into the inside of the body is simplified.

According to the above configuration, since the width of a portion of the slit is smaller than the diameter of the cable by the portion forming the projection, the camera-side cable is not easily removed from the camera support tube, and the workability is gradually improved when the camera support tube is installed.

The present invention is not limited to each of the above-described embodiments, various changes are possible within the range described in claims, and an embodiment which can be obtained by appropriately combining technical means which are respectively described in different embodiments is also included in the technical range of the present invention. Furthermore, by combining the technical means which are respectively described in each embodiment, it is possible to form new technical characteristics.

### INDUSTRIAL APPLICABILITY

The image capturing device is suitable for an endoscopic surgery and the like.

### REFERENCE SIGNS LIST

1 CAMERA SYSTEM FOR MONITORING AN INSIDE OF A BODY

11 CAMERA UNIT (IMAGING UNIT)

12 CAMERA-SIDE CABLE (FIRST CABLE)

13 CAMERA SUPPORT TUBE (SUPPORT TUBE)

14 SUPPORT-TUBE JOINING UNIT (JOINING UNIT)

14d HEAT-CONDUCTIVE PROTRUSION PORTION (JOINING UNIT)

15a CAMERA-SIDE CABLE CONNECTOR (FIRST CONNECTOR)

15b DEVICE-SIDE CABLE CONNECTOR (SECOND CONNECTOR)

16 DEVICE-SIDE CABLE (SECOND CABLE)

17 CAMERA UNIT CONTROL DEVICE

18 DISPLAY (DISPLAY DEVICE)

19 CIRCUIT BOARD

31 CANNULA

131 GROUND CONNECTION LINE (GROUND UNIT, GROUND LINE)

131a CONNECTION-LINE CONNECTOR (GROUND-LINE CONNECTOR)

131b SUPPORT-TUBE SIDE CONNECTION LINE (SUPPORT-TUBE CONNECTION LINE)

132 SUPPORT-TUBE HANDLE UNIT (GRIPPING UNIT)

134 CABLE HOLDER (ACCESSORY FOR SUPPORT TUBE)

191 METAL SPRING (GROUND UNIT)

223 SLIT

404 ELECTRIC-KNIFE COUNTER ELECTRODE PLATE (COUNTER ELECTRODE PLATE)

1. A camera system for monitoring an inside of a body, comprising:
a support tube which has one end portion guided toward an inside of a body and is formed of a conductive material;
a ground unit which has at least a portion formed of a conductive material and causes the support tube to be grounded;
an imaging unit which is joined to the support tube inside the body;
a joining unit that joins the imaging unit and the support tube;
a first cable which is connected to the imaging unit and is drawn toward an outside of the body through the support tube; and
a control system which is provided on the outside of the body, electrically connected to the first cable, and includes at least a display device.

2. The camera system for monitoring an inside of a body, according to claim 1,
wherein the support tube has at least a portion which is non-conductive and is capable of being brought into contact with a body wall when being joined to the imaging unit.

3. The camera system for monitoring an inside of a body, according to claim 1,
wherein a portion of the joining unit, which is brought into contact with the support tube, has at least a part which is formed of a conductive material, and
the ground unit is electrically connected to the support tube, the joining unit, and a grounded circuit board of the imaging unit.

4. The camera system for monitoring an inside of a body, according to claim 1, further comprising:
a second cable which is electrically connected to the control system,
wherein a first connector provided on the first cable is fitted to a second connector provided on the second cable, and
the ground unit is electrically connected to any one of the first connector and the second connector which is grounded.

5. The camera system for monitoring an inside of a body, according to claim 4,
wherein the ground unit is connected to the grounded second connector.

6. The camera system for monitoring an inside of a body, according to claim 4,
wherein the ground unit is a ground line which is capable of being electrically connected to the support tube.

7. The camera system for monitoring an inside of a body, according to claim 6,
wherein the ground line is detachable from the support tube through a ground-line connector or a support-tube connection line which is provided on the support tube, and
the support-tube connection line has such a length that it does not come into contact with a cannula when the support tube is inserted into the cannula having an annular structure in which the support tube is capable of being inserted in the cannula.

8. The camera system for monitoring an inside of a body, according to claim 6, further comprising:
an accessory for the support tube, which is connectable to the ground line and is attachable to the support tube or a body-outside end portion of the cannula having an annular structure in which the support tube is capable of being inserted in the cannula, so as to be brought into contact with the support tube; and
the accessory for the support tube has at least a portion which is formed of a conductive material so as to electrically connect the support tube to the ground line.

9. The camera system for monitoring an inside of a body, according to claim 6,
wherein the ground line is electrically connected to a grounded current feedback terminal of an electric knife or a counter electrode plate attached to a patient.

10. The camera system for monitoring an inside of a body, according to claim 1,
wherein a slit is formed in the support tube so as to enable the first cable to pass from a side surface of the support tube to an inside of the support tube.

11. The camera system for monitoring an inside of a body, according to claim 10,
wherein an edge of the slit includes a portion forming a projection, and
a portion of the slit, which corresponds to the portion forming the projection, has a width narrower than a diameter of the first cable.

12. The camera system for monitoring an inside of a body, according to claim 1,
wherein the support tube has a gripping unit at a body-outside end portion, and
the gripping unit has an outer diameter greater than an inner diameter of the cannula having an annular structure in which the support tube is capable of being inserted in the cannula.

13. A camera system for monitoring an inside of a body, comprising:
a support tube which has one end portion guided toward an inside of a body;
an imaging unit which is joined to the support tube inside the body;
a joining unit that joins the imaging unit and the support tube;
a first cable which is connected to the imaging unit and is drawn toward an outside of the body through the support tube; and
a control system which is provided on the outside of the body, electrically connected to the first cable, and includes at least a display device,
wherein the support tube has a gripping unit at a body-outside end portion, and
the gripping unit has an outer diameter greater than an inner diameter of the cannula having an annular structure in which the support tube is capable of being inserted in the cannula.

14. A camera system for monitoring an inside of a body, comprising:
a support tube which has one end portion guided toward an inside of a body;
an imaging unit which is joined to the support tube inside the body;
a joining unit that joins the imaging unit and the support tube;
a first cable which is connected to the imaging unit and is drawn toward an outside of the body through the support tube; and
a control system which is provided on the outside of the body, electrically connected to the first cable, and includes at least a display device,
wherein a slit is formed in the support tube so as to enable the first cable to pass from a side surface of the support tube to an inside of the support tube,
an edge of the slit includes a portion forming a projection, and
a portion of the slit, which corresponds to the portion forming the projection, has a width narrower than a diameter of the first cable.
